SEQUENCE LISTING (40 sequences)

SEQ ID NO: 1 - protein, 20 aa, Artificial Sequence
  Synthetic
  misc_feature: Linker
PSGQAGAAASESLFVSNHAY

SEQ ID NO: 2 - protein, 7 aa, Artificial Sequence
  Synthetic
  misc_feature: Linker
EASGGPE

SEQ ID NO: 3 - protein, 539 aa, Artificial Sequence
  Synthetic
  misc_feature: Llama161533
MKWVTFISLLFLFSSAYSQVQLVESGGGLVQPGGSLRLSCAASGLTFSSYNMGWFRQAPGQGLEAVASITWSGRDTFYADSVKGRFTISRDNSKNTLYLQMNSLRAEDTAVYYCAANPWPVAAPRSGTYWGQGTLVTVSSSGGGGSGGGGSGGGGSGGGGSGNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGSTSGSGKPGSGEGSTKGQVQLVQSGAEVKKPGSSVKVSCKASGYTFTDYNMHWVRQAPGQGLEWIGYIYPYNGGTGYNQKFKSKATITADESTNTAYMELSSLRSEDTAVYYCARGRPAMDYWGQGTLVTVSSGGGGSGGGGSGGGGSDIQMTQSPSSLSASVGDRVTITCRASESVDNYGISFMNWFQQKPGKAPKLLIYAASNQGSGVPSRFSGSGSGTDFTLTISSLQPDDFATYYCQQSKEVPWTFGQGTKVEIKVDE

SEQ ID NO: 4 - protein, 114 aa, Artificial Sequence
  Synthetic
  misc_feature: Il-15 (human)
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 5 - protein, 630 aa, Artificial Sequence
  Synthetic
  misc_feature: 1615antiHIV
MEVQLVESGGGVVRPGGSLRLSCAASGFTFDDYGMSWVRQAPGKGLEWVSGINWNGGSTGYADSVKGRFTISRDNAKNSLYLQMNSLRAEDTAVYYCARGRSLLFDYWGQGTLVTVSRGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVLPSGQAGAAASESLFVSNHAYNWVNVISDLKKIEDLIQSMHIDATLYTESDVHPSCKVTAMKCFLLELQVISLESGDASIHDTVENLIILANDSLSSNGNVTESGCKECEELEEKNIKEFLQSFVHIVQMFINTSEASGGPEMGWSCIILFLVATATGVHSQVRLSQSGGQMKKPGDSMRISCRASGYEFINCPINWIRLAPGKRPEWMGWMKPRHGAVSYARQLQGRVTMTRDMYSETAFLELRSLTSDDTAVYFCTRGKYCTARDYYNWDFEHWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDK

SEQ ID NO: 6 - DNA, 1568 bp, Artificial Sequence
  Synthetic
  misc_feature: Cam1615PGT121
ccatggagcaggtgcagctggtggagtctgggggaggcttggtgcagcctgggggctctctgagactctcctgtgcagcctctggcctcaccttcagtagctataacatgggctggttccgccaggctccagggcaaggccttgaggctgtagcatctattacctggagtggtcgggacacattctatgcagactccgtgaagggccgattcaccatctccagagacaactccaagaacactctctatctgcaaatgaacagcctgcgcgcggaggacacggccgtttattattgtgctgcaaacccctggccagtggcggcgccacgtagtggcacctactggggccaagggaccctggtcaccgtctcctcaccgtctggtcaggctggtgctgctgctagcgaatctctgttcgtttctaaccacgcttacaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttctgaagcttccggaggtcccgagcagatgcagttacaggagtcgggccccggactggtgaagccttcggaaaccctgtccctcacgtgcagtgtgtctggtgcctccataagtgacagttactggagctggatccggcggtccccagggaagggacttgagtggattgggtatgtccacaaaagcggcgacacaaattacagcccctccctcaagagtcgagtcaacttgtcgttagacacgtccaaaaatcaggtgtccctgagccttgtggccgcgaccgctgcggactcgggcaaatattattgcgcgagaacactgcacgggaggagaatttatggaatcgttgccttcaatgagtggttcacctacttctacatggacgtctggggcaatgggactcaggtcaccgtctcctcaggtggaggcggttcaggcggaggtggctctggcggtggcggatcgtccgatatatctgtggccccaggagagacggccaggatttcctgtggggaaaagagccttggaagtagagctgtacaatggtatcaacacagggccggccaggccccctctttaatcatatataataatcaggaccggccctcagggatccctgagcgattctctggctcccctgactccccttttgggaccacggccaccctgaccatcaccagtgtcgaagccggggatgaggccgactattactgtcatatatgggatagtagagttcccaccaaatgggtcttcggcggagggaccacgctgaccgtgttatagtgactcgagcag

SEQ ID NO: 7 - protein, 517 aa, Artificial Sequence
  Synthetic
  misc_feature: Cam1615PGT121
MEQVQLVESGGGLVQPGGSLRLSCAASGLTFSSYNMGWFRQAPGQGLEAVASITWSGRDTFYADSVKGRFTISRDNSKNTLYLQMNSLRAEDTAVYYCAANPWPVAAPRSGTYWGQGTLVTVSSPSGQAGAAASESLFVSNHAYNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSEASGGPEQMQLQESGPGLVKPSETLSLTCSVSGASISDSYWSWIRRSPGKGLEWIGYVHKSGDTNYSPSLKSRVNLSLDTSKNQVSLSLVAATAADSGKYYCARTLHGRRIYGIVAFNEWFTYFYMDVWGNGTQVTVSSGGGGSGGGGSGGGGSSDISVAPGETARISCGEKSLGSRAVQWYQHRAGQAPSLIIYNNQDRPSGIPERFSGSPDSPFGTTATLTITSVEAGDEADYYCHIWDSRVPTKWVFGGGTTLTVL

SEQ ID NO: 8 - protein, 252 aa, Artificial Sequence
  Synthetic
  misc_feature: PGT121
QMQLQESGPGLVKPSETLSLTCSVSGASISDSYWSWIRRSPGKGLEWIGYVHKSGDTNYSPSLKSRVNLSLDTSKNQVSLSLVAATAADSGKYYCARTLHGRRIYGIVAFNEWFTYFYMDVWGNGTQVTVSSGGGGSGGGGSGGGGSSDISVAPGETARISCGEKSLGSRAVQWYQHRAGQAPSLIIYNNQDRPSGIPERFSGSPDSPFGTTATLTITSVEAGDEADYYCHIWDSRVPTKWVFGGGTTLTVL

SEQ ID NO: 9 - protein, 372 aa, Artificial Sequence
  Synthetic
MGWSCIILFLVATATGVHSSLTQSPGTLSLSPGETAIISCRTSQYGSLAWYQQRPGQAPRLVIYSGSTRAAGIPDRFSGSRWGPDYNLTISNLESGDFGVYYCQQYEFFGQGTKVQVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSEVQLVESGGELVQAGGSLRLSCAASGLTFSSYNMGWFRRAPGKEREFVASITWSGRDTFYADSVKGRFTISRDNAKNTVYLQMSSLKPEDTAVYYCAANPWPVAAPRSGTYWGQGTQVTVSSVDEHHHHHHHHHH

SEQ ID NO: 10 - protein, 20 aa, Mouse
MGWSCIILFLVATATGVHSS

SEQ ID NO: 11 - protein, 125 aa, Artificial Sequence
  Synthetic
EVQLVESGGELVQAGGSLRLSCAASGLTFSSYNMGWFRRAPGKEREFVASITWSGRDTFYADSVKGRFTISRDNAKNTVYLQMSSLKPEDTAVYYCAANPWPVAAPRSGTYWGQGTQVTVSSVDE

SEQ ID NO: 12 - protein, 10 aa, Artificial Sequence
  Synthetic
HHHHHHHHHH

SEQ ID NO: 13 - protein, 249 aa, Artificial Sequence
  Synthetic
MGWSCIILFLVATATGVHSQVRLSQSGGQMKKPGDSMRISCRASGYEFINCPINWIRLAPGKRPEWMGWMKPRHGAVSYARQLQGRVTMTRDMYSETAFLELRSLTSDDTAVYFCTRGKYCTARDYYNWDFEHWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDK

SEQ ID NO: 14 - protein, 19 aa, Mouse
MGWSCIILFLVATATGVHS

SEQ ID NO: 15 - protein, 124 aa, Artificial Sequence
  Synthetic
QVRLSQSGGQMKKPGDSMRISCRASGYEFINCPINWIRLAPGKRPEWMGWMKPRHGAVSYARQLQGRVTMTRDMYSETAFLELRSLTSDDTAVYFCTRGKYCTARDYYNWDFEHWGQGTPVTVS

SEQ ID NO: 16 - protein, 106 aa, Artificial Sequence
  Synthetic
SASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDK

SEQ ID NO: 17 - protein, 372 aa, Artificial Sequence
  Synthetic
MGWSCIILFLVATATGVHSSLTQSPGTLSLSPGETAIISCRTSQYGSLAWYQQRPGQAPRLVIYSGSTRAAGIPDRFSGSRWGPDYNLTISNLESGDFGVYYCQQYEFFGQGTKVQVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSQVQLVESGGGLVQPGGSLRLSCAASGLTFSSYNMGWFRQAPGQGLEAVASITWSGRDTFYADSVKGRFTISRDNSKNTLYLQMNSLRAEDTAVYYCAANPWPVAAPRSGTYWGQGTLVTVSSVDEHHHHHHHHHH

SEQ ID NO: 18 - protein, 122 aa, Artificial Sequence
  Synthetic
QVQLVESGGGLVQPGGSLRLSCAASGLTFSSYNMGWFRQAPGQGLEAVASITWSGRDTFYADSVKGRFTISRDNSKNTLYLQMNSLRAEDTAVYYCAANPWPVAAPRSGTYWGQGTLVTVSS

SEQ ID NO: 19 - protein, 639 aa, Artificial Sequence
  Synthetic
MGWSCIILFLVATATGVHSQVRLSQSGGQMKKPGDSMRISCRASGYEFINCPINWIRLAPGKRPEWMGWMKPRHGAVSYARQLQGRVTMTRDMYSETAFLELRSLTSDDTAVYFCTRGKYCTARDYYNWDFEHWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKEGRGSLLTCGDVEENPGPMGWSCIILFLVATATGVHSSLTQSPGTLSLSPGETAIISCRTSQYGSLAWYQQRPGQAPRLVIYSGSTRAAGIPDRFSGSRWGPDYNLTISNLESGDFGVYYCQQYEFFGQGTKVQVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSQVQLVESGGGLVQPGGSLRLSCAASGLTFSSYNMGWFRQAPGQGLEAVASITWSGRDTFYADSVKGRFTISRDNSKNTLYLQMNSLRAEDTAVYYCAANPWPVAAPRSGTYWGQGTLVTVSSVDEHHHHHHHHHH

SEQ ID NO: 20 - protein, 643 aa, Artificial Sequence
  Synthetic
MKHLWFFLLLVAAPRWVLSQMQLQESGPGLVKPSETLSLTCSVSGASISDSYWSWIRRSPGKGLEWIGYVHKSGDTNYSPSLKSRVNLSLDTSKNQVSLSLVAATAADSGKYYCARTLHGRRIYGIVAFNEWFTYFYMDVWGNGTQVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEEGRGSLLTCGDVEENPGPMAWTFLLLGLLSHCTASVTSDISVAPGETARISCGEKSLGSRAVQWYQHRAGQAPSLIIYNNQDRPSGIPERFSGSPDSPFGTTATLTITSVEAGDEADYYCHIWDSRVPTKWVFGGGTTLTVLGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECSGGGGSGGGGSQVQLVESGGGLVQPGGSLRLSCAASGLTFSSYNMGWFRQAPGQGLEAVASITWSGRDTFYADSVKGRFTISRDNSKNTLYLQMNSLRAEDTAVYYCAANPWPVAAPRSGTYWGQGTLVTVSSVDEHHHHHHHHHH

SEQ ID NO: 21 - protein, 207 aa, Artificial Sequence
  Synthetic
  misc_feature: HIV antibody light chain
LTQSPGTLSLSPGETAIISCRTSQYGSLAWYQQRPGQAPRLVIYSGSTRAAGIPDRFSGSRWGPDYNLTISNLESGDFGVYYCQQYEFFGQGTKVQVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 22 - protein, 230 aa, Artificial Sequence
  Synthetic
  misc_feature: HIV antibody Heavy chain
QVRLSQSGGQMKKPGDSMRISCRASGYEFINCPINWIRLAPGKRPEWMGWMKPRHGAVSYARQLQGRVTMTRDMYSETAFLELRSLTSDDTAVYFCTRGKYCTARDYYNWDFEHWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDK

SEQ ID NO: 23 - protein, 10 aa, Artificial Sequence
  Synthetic
GGGGSGGGGS

SEQ ID NO: 24 - protein, 547 aa, Artificial Sequence
  Synthetic
MKWVTFISLLFLFSSAYSQVQLVESGGGLVQPGGSLRLSCAASGLTFSSYNMGWFRQAPGQGLEAVASITWSGRDTFYADSVKGRFTISRDNSKNTLYLQMNSLRAEDTAVYYCAANPWPVAAPRSGTYWGQGTLVTVSSSGGGGSGGGGSGGGGSGGGGSGNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGSTSGSGKPGSGEGSTKGQVQLVQSGGQMKKPGESMRISCRASGYEFIDCTLNWIRLAPGKRPEWMGWLKPRGGAVNYARPLQGRVTMTRDVYSDTAFLELRSLTVDDTAVYFCTRGKNCDYNWDFEHWGRGTPVIVSSGGGGSGGGGSGGGGSEIVLTQSPGTLSLSPGETAIISCRTSQYGSLAWYQQRPGQAPRLVIYSGSTRAAGIPDRFSGSRWGPDYNLTISNLESGDFGVYYCQQYEFFGQGTKVQVDIKRVDEHHHHHHHHHH

SEQ ID NO: 25 - protein, 119 aa, Artificial Sequence
  Synthetic
QVQLVQSGGQMKKPGESMRISCRASGYEFIDCTLNWIRLAPGKRPEWMGWLKPRGGAVNYARPLQGRVTMTRDVYSDTAFLELRSLTVDDTAVYFCTRGKNCDYNWDFEHWGRGTPVIV

SEQ ID NO: 26 - protein, 104 aa, Artificial Sequence
  Synthetic
EIVLTQSPGTLSLSPGETAIISCRTSQYGSLAWYQQRPGQAPRLVIYSGSTRAAGIPDRFSGSRWGPDYNLTISNLESGDFGVYYCQQYEFFGQGTKVQVDIKR

SEQ ID NO: 27 - protein, 104 aa, Artificial Sequence
  Synthetic
LTQSPGTLSLSPGETAIISCRTSQYGSLAWYQQRPGQAPRLVIYSGSTRAAGIPDRFSGSRWGPDYNLTISNLESGDFGVYYCQQYEFFGQGTKVQVDIKRTVA

SEQ ID NO: 28 - protein, 103 aa, Artificial Sequence
  Synthetic
APSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 29 - protein, 571 aa, Artificial Sequence
  Synthetic
MKWVTFISLLFLFSSAYSQVQLVESGGGLVQPGGSLRLSCAASGLTFSSYNMGWFRQAPGQGLEAVASITWSGRDTFYADSVKGRFTISRDNSKNTLYLQMNSLRAEDTAVYYCAANPWPVAAPRSGTYWGQGTLVTVSSSGGGGSGGGGSGGGGSGGGGSGNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGSTSGSGKPGSGEGSTKGSGEGSTKGEVQLVESGGGLVKPGGSLRLSCSASGFDFDNAWMTWVRQPPGKGLEWVGRITGPGEGWSVDYAAPVEGRFTISRLNSINFLYLEMNNLRMEDSGLYFCARTGKYYDFWSGYPPGEEYFQDWGRGTLVTVSSGGGGSGGGGSGGGGSSYELTQETGVSVALGRTVTITCRGDSLRSHYASWYQKKPGQAPILLFYGKNNRPSGVPDRFSGSASGNRASLTISGAQAEDDAEYYCSSRDKSGSRLSVFGGGTKLTVLLVDEHHHHHHHHHH

SEQ ID NO: 30 - protein, 137 aa, Artificial Sequence
  Synthetic
SGEGSTKGEVQLVESGGGLVKPGGSLRLSCSASGFDFDNAWMTWVRQPPGKGLEWVGRITGPGEGWSVDYAAPVEGRFTISRLNSINFLYLEMNNLRMEDSGLYFCARTGKYYDFWSGYPPGEEYFQDWGRGTLVTV

SEQ ID NO: 31 - protein, 110 aa, Artificial Sequence
  Synthetic
SYELTQETGVSVALGRTVTITCRGDSLRSHYASWYQKKPGQAPILLFYGKNNRPSGVPDRFSGSASGNRASLTISGAQAEDDAEYYCSSRDKSGSRLSVFGGGTKLTVLL

SEQ ID NO: 32 - protein, 505 aa, Artificial Sequence
  Synthetic
MEQVQLVESGGGLVQPGGSLRLSCAASGLTFSSYNMGWFRQAPGQGLEAVASITWSGRDTFYADSVKGRFTISRDNSKNTLYLQMNSLRAEDTAVYYCAANPWPVAAPRSGTYWGQGTLVTVSSPSGQAGAAASESLFVSNHAYNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSEASGGPEQVQLVQSGAEVKKPGASVKVSCKASGYSFTGYTMNWVRQAPGQGLEWMGLITPYNGASSYNQKFRGKATMTVDTSTSTVYMELSSLRSEDTAVYYCARGGYDGRGFDYWGQGTLVTVSSGGGGSGGGGSSGGGSDIQMTQSPSSLSASVGDRVTITCSASSSVSYMHWYQQKSGKAPKLLIYDTSKLASGVPSRFSGSGSGTDFTLTISSLQPEDFATYYCQQWSKHPLTFGQGTKLEIK

SEQ ID NO: 33 - protein, 240 aa, Artificial Sequence
  Synthetic
QVQLVQSGAEVKKPGASVKVSCKASGYSFTGYTMNWVRQAPGQGLEWMGLITPYNGASSYNQKFRGKATMTVDTSTSTVYMELSSLRSEDTAVYYCARGGYDGRGFDYWGQGTLVTVSSGGGGSGGGGSSGGGSDIQMTQSPSSLSASVGDRVTITCSASSSVSYMHWYQQKSGKAPKLLIYDTSKLASGVPSRFSGSGSGTDFTLTISSLQPEDFATYYCQQWSKHPLTFGQGTKLEIK

SEQ ID NO: 34 - protein, 774 aa, Artificial Sequence
  Synthetic
MEQVQLVESGGGLVQPGGSLRLSCAASGLTFSSYNMGWFRQAPGQGLEAVASITWSGRDTFYADSVKGRFTISRDNSKNTLYLQMNSLRAEDTAVYYCAANPWPVAAPRSGTYWGQGTLVTVSSPSGQAGAAASESLFVSNHAYNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSEASGGPEQVQLVQSGAEVKKPGASVKVSCKASGYSFTGYTMNWVRQAPGQGLEWMGLITPYNGASSYNQKFRGKATMTVDTSTSTVYMELSSLRSEDTAVYYCARGGYDGRGFDYWGQGTLVTVSSGGGGSGGGGSSGGGSDIQMTQSPSSLSASVGDRVTITCSASSSVSYMHWYQQKSGKAPKLLIYDTSKLASGVPSRFSGSGSGTDFTLTISSLQPEDFATYYCQQWSKHPLTFGQGTKLEIKEPKSSDKTHTSPPSPELDIVLSQSPAIMSASPGEKVTISCSASSSVSYMYWYQQKPGSSPKPWIYRTSNLASGVPARFSGSGSGTSYSLTISSMEAEDAATYYCQQYHSYPPTFGAGTKLELKSSGGGGSGGGGGGSSRSSLEVKLVESGPELKKPGETVKISCKASGYTFTDYSMHWVNQAPGKGLKWMGWINTETGEPSYADDFKGRFAFSLETSASTAYLQINNLKNEDTATYFCATDYGDYFDYWGQGTTLTVSSAKTTPPSVTS

SEQ ID NO: 35 - protein, 509 aa, Artificial Sequence
  Synthetic
QVQLVQSGAEVKKPGASVKVSCKASGYSFTGYTMNWVRQAPGQGLEWMGLITPYNGASSYNQKFRGKATMTVDTSTSTVYMELSSLRSEDTAVYYCARGGYDGRGFDYWGQGTLVTVSSGGGGSGGGGSSGGGSDIQMTQSPSSLSASVGDRVTITCSASSSVSYMHWYQQKSGKAPKLLIYDTSKLASGVPSRFSGSGSGTDFTLTISSLQPEDFATYYCQQWSKHPLTFGQGTKLEIKEPKSSDKTHTSPPSPELDIVLSQSPAIMSASPGEKVTISCSASSSVSYMYWYQQKPGSSPKPWIYRTSNLASGVPARFSGSGSGTSYSLTISSMEAEDAATYYCQQYHSYPPTFGAGTKLELKSSGGGGSGGGGGGSSRSSLEVKLVESGPELKKPGETVKISCKASGYTFTDYSMHWVNQAPGKGLKWMGWINTETGEPSYADDFKGRFAFSLETSASTAYLQINNLKNEDTATYFCATDYGDYFDYWGQGTTLTVSSAKTTPPSVTS

SEQ ID NO: 36 - protein, 610 aa, Artificial Sequence
  Synthetic
MEVQLVESGGGVVRPGGSLRLSCAASGFTFDDYGMSWVRQAPGKGLEWVSGINWNGGSTGYADSVKGRFTISRDNAKNSLYLQMNSLRAEDTAVYYCARGRSLLFDYWGQGTLVTVSRGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVLPSGQAGAAASESLFVSNHAYNWVNVISDLKKIEDLIQSMHIDATLYTESDVHPSCKVTAMKCFLLELQVISLESGDASIHDTVENLIILANDSLSSNGNVTESGCKECEELEEKNIKEFLQSFVHIVQMFINTSEASGGPEQVQLVQSGAEVKKPGASVKVSCKASGYSFTGYTMNWVRQAPGQGLEWMGLITPYNGASSYNQKFRGKATMTVDTSTSTVYMELSSLRSEDTAVYYCARGGYDGRGFDYWGQGTLVTVSSGGGGSGGGGSSGGGSDIQMTQSPSSLSASVGDRVTITCSASSSVSYMHWYQQKSGKAPKLLIYDTSKLASGVPSRFSGSGSGTDFTLTISSLQPEDFATYYCQQWSKHPL

SEQ ID NO: 37 - protein, 558 aa, Artificial Sequence
  Synthetic
MKWVTFISLLFLFSSAYSQVQLVESGGGLVQPGGSLRLSCAASGLTFSSYNMGWFRQAPGQGLEAVASITWSGRDTFYADSVKGRFTISRDNSKNTLYLQMNSLRAEDTAVYYCAANPWPVAAPRSGTYWGQGTLVTVSSSGGGGSGGGGSGGGGSGGGGSGNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGSTSGSGKPGSGEGSTKGQHLVQSGTQVKKPGASVRISCQASGYSFTDYVLHWWRQAPGQGLEWMGWIKPVYGARNYARRFQGRINFDRDIYREIAFMDLSGLRSDDTALYFCARDGSGDDTSWHLDPWGQGTLVIVSAASTKGGGGGSGGGGSGGGGSDIQMTQSPSSLSASVGDRVTITCQAGQGIGSSLQWYQQKPGKAPKLLVHGASNLHRGVPSRFSGSGFHTTFSLTISGLQRDDFATYFCAVLEFFGPGTKVEIKRTVAAPSVDEHHHHHHHHHH

SEQ ID NO: 38 - protein, 122 aa, Artificial Sequence; identical to SEQ ID NO: 18
  Synthetic

SEQ ID NO: 39 - protein, 125 aa, Artificial Sequence
  Synthetic
QHLVQSGTQVKKPGASVRISCQASGYSFTDYVLHWWRQAPGQGLEWMGWIKPVYGARNYARRFQGRINFDRDIYREIAFMDLSGLRSDDTALYFCARDGSGDDTSWHLDPWGQGTLVIVSAASTK

SEQ ID NO: 40 - protein, 110 aa, Artificial Sequence
  Synthetic
DIQMTQSPSSLSASVGDRVTITCQAGQGIGSSLQWYQQKPGKAPKLLVHGASNLHRGVPSRFSGSGFHTTFSLTISGLQRDDFATYFCAVLEFFGPGTKVEIKRTVAAPS

(19) United States
(12) Patent Application Publication
(10) Pub. No.: US 2022/0363737 A1
(43) Pub. Date: Nov. 17, 2022

(54) NK ENGAGER COMPOUNDS THAT BIND VIRAL ANTIGENS AND METHODS OF USE

(71) Applicant: Regents of the University of Minnesota, Minneapolis, MN (US)

(72) Inventors: Jeffrey S. Miller, Minneapolis, MN (US); Martin Felices, Minneapolis, MN (US); Todd Lenvik, Minneapolis, MN (US); Daniel A. Vallera, Minneapolis, MN (US)

(21) Appl. No.: 17/762,354

(22) PCT Filed: Sep. 25, 2020
(86) PCT No.: PCT/US2020/052671

§ 371 Date: Mar. 21, 2022

Related U.S. Application Data
(60) Provisional application No. 62/906,660, filed on Sep. 26, 2019.

(51) Int. Cl. C07K 16/10; A61P 31/18; C07K 14/54; C07K 16/28

(52) U.S. Cl. CPC C07K 16/1045; A61P 31/18; C07K 14/5443; C07K 16/283; C07K 2317/569; C07K 2317/22; A61K 2039/505

(57) ABSTRACT

This disclosure describes compounds that engage NK cells and methods of using the compounds. Generally, the compound includes an NK engaging domain, a targeting domain that selectively binds to a target cell, and an NK activating domain operably linking the NK engaging domain and the targeting domain. In an illustrative embodiment, the targeting domain selectively binds to an HIV antigen.

### CROSS-REFERENCE TO RELATED APPLICATIONS

This application claims benefit of priority under 35 U.S.C. §119(e) of U.S. Ser. No. 62/906,660, filed Sep. 26, 2019, the entire contents of which is incorporated herein by reference in its entirety.

### STATEMENT OF GOVERNMENT SUPPORT

This invention was made with government support under CA111412 and CA065493, awarded by the National Institutes of Health, and under CA036725, CA072669, CA077598 and CA197292, awarded by the National Cancer Institute. The government has certain rights in the invention.

### INCORPORATION OF SEQUENCE LISTING

The material in the accompanying sequence listing is hereby incorporated by reference into this application. The accompanying sequence listing text file, name GTBI02130_1WO_Sequence_Listing.txt, was created on Sep. 25, 2020, and is 96 kb. The file can be accessed using Microsoft Word on a computer that uses Windows OS.

### BACKGROUND INFORMATION

Natural killer (NK) cells are cytotoxic lymphocytes of the innate immune system capable of immune surveillance. NK cells express CD16, an activating receptor that binds to the Fc portion of IgG antibodies and is involved in antibody-dependent cell-mediated cytotoxicity (ADCC). NK cells are regulated by IL-15. IL-15 can induce increased antigen-dependent cytotoxicity, lymphokine-activated killer activity, and/or mediate cytokine responses. NK cells can be activated to stimulate an immune response for treating cancer and infections as an NK-cell-based immunotherapy.

While advancements in efficacy and use of anti-retroviral drugs have substantially ameliorated the health and longevity of HIV-infected individuals, these drugs are merely a stop-gap to prevent progression to AIDS and to limit further transmission of the virus. Despite the use of anti-virals to suppress HIV replication, infected individuals retain reservoirs of latently HIV-infected cells that, upon cessation of anti-retroviral therapy, could reactivate and re-establish an active infection. A curative solution necessitates the reactivation and subsequent destruction of these latently infected cells. The antibody response to HIV infection, while present, is generally ineffective due to the high rate of mutation of the virus which can rapidly eliminate epitopes recognized by the generated antibodies. However, in recent years a variety of HIV-specific antibodies have been identified in infected individuals which have strong neutralizing effects but a poor ability to elicit antibody dependent cell-mediated cytotoxicity (ADCC).

### SUMMARY OF THE INVENTION

The present invention provides compounds for activating NK cells to stimulate an immune response for treating cancer and viral infections. The inventors have designed bi-and tri-specific killer engagers (BiKE and TriKE™) composed of a CD16 engager linked by an IL-15 molecule. As used herein, the compounds of the invention may be referred to as a 16/15/X TriKE, wherein X represents a targeting domain. The X targeting domain may be directed to as examples, viral antigens, cancer cell antigens and the like.

In a particular embodiment, an antibody construct was constructed to take advantage of the broad specificity of these antibodies to target HIV while redirecting NK cell killing specifically to actively replicating infected cells though its recognition of membrane expressed Env and triggering NK cell degranulation though the low affinity Fc receptor, CD16. The addition of IL-15 as a linker further activates NK cells thereby enhancing their response. IL-15 has also been identified as a potential reactivator of latently infected cells. Initial studies show enhanced NK cell cytokine production and killing of infected targets expressing HIV-Env when incubated with the HIV-specific constructs of the invention.

In one embodiment, the invention provides a compound having an NK engaging domain including a moiety that selectively binds to CD16 or NKG2c; an NK activating domain operably linked to the NK engaging domain including IL-15 or a functional fragment thereof; and a targeting domain that selectively binds to a viral antigen and is operably linked to the NK activating domain and the NK engaging domain. In some embodiments, the CD16 is CD16a. In some embodiments, the viral antigen is present on an infected cell. In some embodiments, the viral antigen is derived from HIV, CMV, HPV, HCV, or an adenovirus. In some embodiments, the viral antigen is derived from HIV. In some embodiments, the NK engaging domain moiety includes an antibody or a binding fragment thereof or a nanobody. In some embodiments, the antibody fragment includes an scFv, a F(ab)2, or a Fab. In some embodiments, the antibody or a binding fragment thereof or the nanobody is human. In some embodiments, the antibody or a binding fragment thereof or the nanobody is camelid. In some embodiments, the IL-15 has an amino acid sequence of SEQ ID NO: 4 or a functional variant thereof. In some embodiments, the functional variant of IL-15 includes an N72D or N72A amino acid substitution as compared to SEQ ID NO: 4. In some embodiments, the targeting domain moiety includes an antibody or a binding fragment thereof or a nanobody. In some embodiments, the antibody binding fragment includes an scFv, a F(ab)2, or a Fab. In some embodiments, the NK engaging domain includes CD16, the NK activating domain includes IL-15, and the targeting domain selectively binds to a viral antigen derived from HIV. In some embodiments, the NK engaging domain includes CD16a, the NK activating domain includes IL-15, and the targeting domain selectively binds to a viral antigen derived from HIV. In some embodiments, the NK engaging domain includes NKG2c, the NK activating domain includes IL-15, and the targeting domain selectively binds to a viral antigen derived from HIV. In some embodiments, the compounds described herein include at least one flanking sequence linking two of the domains. In some embodiments, the compounds described herein further include a second flanking sequence linking the two linked domains with the third domain. In some embodiments, the flanking sequences flank the NK activating domain. In some embodiments, a first flanking sequence is C-terminal to the NK engaging domain and wherein a second flanking sequence is N-terminal to the anti-viral targeting domain. In some embodiments, the compounds described herein further include a second targeting domain. In some embodiments, the compounds described herein further includes a second NK engaging domain. In some embodiments, the compounds described herein further includes a second NK activating domain.

Also provided are compositions including a compound described herein and a pharmaceutically acceptable carrier.

Further, in some embodiments, are methods including administering to a subject a compound described herein in an amount effective to induce NK-mediated killing of a target cell. In some embodiments, the target cell is infected with a virus. In some embodiments, the virus is HIV, CMV, HPV, HCV, or an adenovirus. In some embodiments, the virus is HIV.

In some embodiments, provided are methods for stimulating expansion of NK cells in vivo, the methods including administering to a subject an amount of a compound described herein effective to stimulate expansion of NK cells in the subject. In some embodiments, the subject is infected with a virus. In some embodiments, the virus is HIV, CMV, HPV, HCV, or an adenovirus. In one aspect the virus is HIV.

In some embodiments, provided herein are methods of treating viral infection in a subject, the method including administering to the subject an amount of a compound described herein effective for treating the viral infection. In some embodiments, the subject is infected with HIV, CMV, HPV, HCV, or an adenovirus. In one embodiment the subject is infected with HIV.

In some embodiments, provided herein, are compounds including: a T cell engaging domain having a moiety that selectively binds to CD3; a T cell activating domain operably linked to the T cell engaging domain including a cytokine of the IL-2 family or a functional fragment thereof; and a targeting domain that selectively binds to a viral antigen and is operably linked to the T cell activating domain and the T cell engaging domain. In some embodiments, the viral antigen is present on an infected cell. In some embodiments, the viral antigen is derived from HIV, CMV, HPV, HCV, or an adenovirus. In some embodiments, the viral antigen is derived from HIV.

In some embodiments, provided herein, are methods including administering to a subject a compound described herein in an amount effective to induce T-cell mediated killing of a target cell. In some embodiments, the target cell is infected with a virus. In some embodiments, the virus is HIV, CMV, HPV, HCV, or an adenovirus. In one embodiment, the virus is HIV and the target is HIV Env protein (e.g., gp120).

In some embodiments, provided herein, are methods for stimulating expansion of T cells in vivo, the methods including administering to a subject an amount of compound described herein effective to stimulate expansion of T cells in the subject. In some embodiments, the subject is infected with a virus. In some embodiments, the virus is HIV, CMV, HPV, HCV, or an adenovirus. In some embodiments, the virus is HIV.

In one embodiment, the invention provides methods of treating mesothelioma including administering to a subject a compound including an amino acid sequence of SEQ ID NO:32 or 36 which includes the target domain of SEQ ID NO:33 in an amount effective to induce NK-mediated killing of a target cell expressing mesothelin.

In some embodiments, provided herein, are methods of making a compound described herein including (i) co-transfecting into mammalian cells a first polynucleotide having a nucleotide sequence encoding an amino acid sequence including an immunoglobulin heavy chain and a second polynucleotide having a nucleotide sequence encoding an amino acid sequence including an immunoglobulin light chain, and (ii) collecting a supernatant from the mammalian cells. In some embodiments, the viral antigen is derived from HIV, CMV, HPV, HCV, or an adenovirus. In some embodiments, the viral antigen is derived from HIV. In some embodiments, the viral antigen is Env. For example, light and heavy chains for anti-HIV antibodies include SEQ ID NOs: 21 and 22; 31 and 30; 26 and 25; and 40 and 39 respectively.

In some embodiments, provided herein, is an isolated DNA sequence encoding the amino acid sequences described herein.

The invention also provides pharmaceutical compositions comprising the TriKE compounds described herein. For example, the invention provides a pharmaceutical composition comprising SEQ ID NO:5, 7, 24, 29, 32, 34, 36 or 37 in a pharmaceutically acceptable carrier. In one embodiment, the invention provides a method of treating a subject by administering a pharmaceutical composition comprising SEQ ID NO: 5, 7, 24, 29, 32, 34, 36 or 37 in a pharmaceutically acceptable carrier.

In one embodiment, the disclosure provides a method of making an invention compound including co-transfecting into mammalian cells a first polynucleotide comprising a nucleotide sequence encoding an amino acid sequence comprising an immunoglobulin heavy chain of SEQ ID NO:22, 25, 30 or 39 and a second polynucleotide comprising a nucleotide sequence encoding an amino acid sequence comprising an immunoglobulin light chain of SEQ ID NO:21, 26, 31 or 40, respectively; and collecting a supernatant from the mammalian cells, wherein the resulting compound binds to a viral antigen. In one aspect, the viral antigen is an HIV antigen.

In one aspect, the invention provides an isolated DNA sequence encoding the amino acid sequences of SEQ ID NOs: 21, 22, 25, 26, 30, 31, 39 or 40. In another aspect, the invention provides a pharmaceutical composition comprising SEQ ID NO:5, 7, 24, 29, 32, 34, 36 and 37 in a pharmaceutically acceptable carrier. In a further aspect, the invention provides a method of treating a subject comprising administering to the subject a pharmaceutical composition comprising SEQ ID NO:5, 7, 24, 29, 32, 34, 36 and 37 in a pharmaceutically acceptable carrier. In a further aspect, the invention provides a method of treating a subject having or being at risk for developing AIDS, comprising administering to the subject a pharmaceutical composition comprising SEQ ID NO:5, 7, 24, 29, and 37.

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIGS. 1A-1C. CD16 nanobody was derived from a published llama nanobody (GeneBank sequence EF561291). The CD16 nanobody was spliced to CD19 to test the ability of this CD16 engager to drive NK cell killing. (A) The CD16 nanobody showed cytolytic NK activity similar to rituximab-mediated killing in a chromium release assay with CD19+Raji targets. (B) The CD16 CDRs were cloned into a humanized camelid scaffold in order to generate HuEF91, a humanized CD16 engager. HuEF91 binding was equivalent to CD16scFv binding, indicating that the humanized HuEF91 did not hinder the specificity of the molecule. (C) The llama161533 TriKE (SEQ ID NO:3) is capable of expanding NK cells.

FIG. 2. Map of CAM1615PGT121 (SEQ ID NO:6-7).

FIG. 3. CAM1615PGT121 nucleotide sequence (SEQ ID NO:6). Lower case and capital letters are used to show domain structure of the nucleic acid sequence as further illustrated in FIG. 4.

FIG. 4. CAM16, hma linker, IL15 WT, EASGGPE linker, PGT121, and stop sequences of the CAM1615PGT121 nucleotide sequence (SEQ ID NO:6). Lower case and capital letters are used to show domain structure of the nucleic acid sequence.

FIG. 5. CAM1615PGT121 amino acid sequence (SEQ ID NO:7). The PGT121 amino acid sequence (SEQ ID NO:8) is shown.

FIG. 6. Two plasmids were co-transfected into mammalian cells to produce a Fab based antibody. Amino acid sequences of proteins generated from plasmid one comprising humanized camelid anti-CD16. (SEQ ID NO:16, 17).

FIG. 7. Sequence of two proteins expressed from a single plasmid using a 2A self-cleaving peptide. (SEQ ID NO:19).

FIG. 8. Amino acid sequences of CAM16_IL15_12A12scFv (HIV TriKE) (SEQ ID NO:37-40).

FIG. 9. Amino acid sequences of CAM16_IL15_VLCO1_scFV_TriKE (SEQ ID NO:24).

FIG. 10. Amino acid sequences of CAM16_IL15 10E8_scFV_TriKE (SEQ ID NO:29).

FIGS. 11A-11B. Structure and function of HIV-specific BiKEs and TriKEs. FIG. 11(A) Shown is a schematic illustrating the origin of of the components for the initial bi-specific HIV-targeting construct comprised of an anti-CD16 short-chain variable fragment linked to a Fab derived from the HIV broadly neutralizing antibody (bnAb) VRC01. FIG. 11(B) Schematic and proposed function of an HIV bi- and tri-specific killer engager (BiKE and TriKE, respectively).

FIGS. 12A-12C. HIV-Env specific BiKE binds CD16-expressing NK cells, HIV-infected cell lines, and induces and HIV-specific NK cell response. FIG. 12 (A) Healthy donor purified peripheral blood NK cells were stained for CD16, streptavidin control or His-tagged BiKE with biotinylated anti-His and fluorochrome conjugated streptavidin. The BiKE binds NK cells reflective of CD16 expression. FIG. 12 (B) Uninfected CD4-expressing HeLa cells or HeLa-CD4 infected with HIV were stained with HIV-Env BiKE. The BiKE specifically bound the infected HeLa-CD4 but not the uninfected HeLa-CD4 demonstrating specificity of the BiKE for cells expressing HIV envelope. FIG. 12 (C) Purified healthy donor NK cells were incubated with infected or uninfected HeLa-CD4 cells with and without HIV-Env BiKE. K562 cells and Rajis with Rituxin were used as controls.

FIGS. 13A-13C. HIV-Env BiKE specifically binds primary infected T-cell lines and mediates NK cell killing. FIG. 13 (A) Two HIV-infected T-cell lines, H9 HIV-IIIB and ACH-2, or their uninfected counterparts, H9 and CEM CD4, were intracellularly stained for HIV capsid protein to confirm active HIV replication. FIG. 13 (B) The same infected and uninfected T-cell lines were stained with the His-tagged HIV-Env BiKE and biotinylated anti-His+ streptavidin or the secondary alone. BiKE showed no binding to the uninfected T-cell lines but bound both infected clones demonstrating a specificity for actively infected T-cells. FIG. 13 (C) Purified NK cells from healthy donors were co-cultured with uninfected or HIV-infected T-cell lines with and without HIV-Env BiKE and assessed for NK degranulation (CD107a) and IFNγ production.

FIGS. 14A-14C. IL-15 containing HIV-TriKE activates immune subsets and induces viral transcription in latently infected primary and T-cell lines. (A) Peripheral blood mononuclear cells were incubated with equimolar rhlL-15 or IL-15 containing HIV-TriKE for 16 hours. NK and T-cell subsets were evaluated by flow cytometry for activation by CD69 expression. FIG. 14 (B) The latently infected human CD4+ T-cell line, ACH-2, was incubated for 48 hours in the presence of 10 nM PMA, 10 ng/mL rhIL-15 or equimolar IL-15 containing TriKE. Cells were then washed and intracellularly stained for HIV-gag (p24). Both IL-15 alone and TriKE induced significant viral reactivation as shown by p24 expression. FIG. 14 (C) Purified CD4+memory T-cells were isolated from anti-retroviral treated, HIV-infected patients and cultured with rhlL-15, the IL-15 Superagonist, Nant-803, or IL-15 containing TriKE. Each condition was incubated with or without the HDAC inhibitor, SAHA for 72 hours. Cells were then harvested, and a nested PCR reaction was done to identify HIV mRNA.

FIG. 15. Solid tumor targeting by second generation TriKE molecules via a number of antigens. NCI-H460: Lung carcinoma (Large cell lung cancer); NCI-H322: Bronchoaveloar carcinoma (Cervical node metastasis); CSPG4: Chondroitin Sulfate Proteoglycan 4; SS1: Mesothelin.

FIGS. 16A-16D. 51 Chromium release assays were performed with several different new TriKEs to show that any scFv that targets cancer cells can be made into functional TriKEs. (A) EpCAM+CD133+NG2+ non-small cell lung cancer NCI-H460 cells plus NK cells were incubated with 1615EPCAM133 TriKE or 1615NG2 TriKE (neuron glial antigen 2 or CSPG4). Both 1615NG2 and 1615EpCAM133 had activity at several different E:T ratios (20:1, 10:1, and 5:1). (B) Mesothelin+EpCAM-CD133-NG2 MDA-435A melanoma cells were incubated with 1615EPCAM TriKE or the 1615Meso TriKE (SEQ ID NO:36) TriKE. Only 1615Meso had activity. (C) Mesothelin+NG2+ ovarian cancer cells (Ovcar3 cells) were incubated with 1615NG2 TriKE or 1615SS1 TriKE. 1615Meso and 1615NG2 had activity. (D)

Raji cells were cultured with NK cells and studied in 51Cr release assays. TriKE 16152219 (SEQ ID NO:12) simultaneously targets the B cell markers CD19 and CD22. Only 16152219, 162219, and Rituximab killed the CD22+ CD19+ targets. The controls did not.

FIG. 17. TriKE sequence (underlined) of SEQ ID NO:3 (llama161533 TriKE).

FIG. 18. CAM1615SS1(mesothelin) amino acid sequence (SEQ ID NO:32). Underlining shows the TriKE sequence. SS1 (mesothelin) scFV antibody fragment is shown (SEQ ID NO:33).

FIG. 19. PGT121Fab was generated using a 2A self-cleaving peptide. In alternative embodiments, a two-plasmid system or an IRES can be used, for example.

FIGS. 20A-20B. FIGS. 20A and 20B Co-transfection of two plasmids into mammalian cells to produce a Fab based antibody. Two separate plasmids (plasmid one and plasmid two) were co-transfected into Expi-Cho-S cells for TriKE generation. Amino acid sequences comprising non-humanized camelid anti-CD16 are shown.

### DETAILED DESCRIPTION OF THE INVENTION

Natural killer (NK) cells are cytotoxic lymphocytes of the innate immune system capable of immune surveillance. Like cytotoxic T cells, NK cells deliver a store of membrane penetrating and apoptosis-inducing granzyme and perforin granules. Unlike T cells, NK cells do not require antigen priming and recognize targets by engaging activating receptors in the absence of MEW recognition.

NK cells express CD16, an activation receptor that binds to the Fc portion of IgG antibodies and is involved in antibody-dependent cell-mediated cytotoxicity (ADCC). NK cells are regulated by IL-15, which can induce increased antigen-dependent cytotoxicity, lymphokine-activated killer activity, and/or mediate interferon (IFN), tumor-necrosis factor (TNF) and/or granulocyte-macrophage colony-stimulating factor (GM-CSF) responses. All of these IL-15-activated functions contribute to improved cancer defense.

The present disclosure describes multi-specific therapeutic compounds that can be a Trispecific Killer Engager compound (TriKEs). TriKEs have three separate binding regions: an NK cell engaging domain that binds to an NK cell (e.g., CD16), an NK activating domain that includes a cytokine or a functional fragment thereof that binds to a receptor for that cytokine, and a targeting domain that binds a marker present on a target cell (e.g., a cancer cell). The design and production of TriKEs are broadly described in, for example, U.S. Patent Application Publication No. US2018/0282386, incorporated by reference in its entirety. TriKEs offer the advantage of combining an antibody-dependent cellular cytotoxicity (ADCC)-facilitating moiety and an expansion-related moiety (IL-15) on the same molecule.

Therapeutically, adoptive transfer of NK cells can, for example, induce remission in patients with refractory acute myeloid leukemia (AML) when combined with lymphodepleting chemotherapy and IL-2 to stimulate survival and in vivo expansion of NK cells. This therapy can be limited by lack of antigen specificity and IL-2-mediated induction of regulatory T (Treg) cells that suppress NK cell proliferation and function. Generating a reagent that drives NK cell antigen specificity, expansion, and/or persistence, while bypassing the negative effects of Treg inhibition, can enhance NK-cell-based immunotherapies.

While advancements in efficacy and use of anti-retroviral drugs have substantially ameliorated the health and longevity of HIV-infected individuals, these drugs are merely a stop-gap to prevent progression to AIDS and to limit further transmission of the virus. Despite the use of antiretrovirals to suppress HIV replication, infected individuals retain reservoirs of latently HIV-infected cells that, upon cessation of anti-retroviral therapy, could reactivate and re-establish an active infection. A curative solution necessitates the reactivation and subsequent destruction of these latently infected cells. The antibody response to HIV infection, while present, is generally ineffective due to the high rate of mutation of the virus which can rapidly eliminate epitopes recognized by the generated antibodies. However, in recent years a variety of HIV-specific antibodies have been identified in infected individuals which have strong neutralizing effects but a poor ability to elicit antibody dependent cell-mediated cytotoxicity (ADCC).

Thus, the invention addresses these issues by providing bi- and tri-specific natural killer cell engagers (BiKE and TriKE) composed of a short-chain variable fragment derived from a broadly-neutralizing antibody (bnAb) against HIV-Env and a CD16 engager linked by an IL-15 molecule. The purpose of this tri-specific antibody construct is to utilize the broad specificity of these antibodies to target HIV while redirecting NK cell killing specifically to actively replicating infected cells though its recognition of membrane expressed Env and triggering NK cell degranulation though the low affinity Fc receptor, CD16. The addition of IL-15 as a linker further activates NK cells thereby enhancing their response. IL-15 has also been identified as a potential reactivator of latently infected cells. Initial studies from our lab show enhanced NK cell cytokine production and killing of infected targets expressing HIV-Env when incubated with the HIV-specific constructs. PBMC from healthy donors incubated with the TriKE showed marked increases in immune cell activation in NK, CD4 and CD8 subsets, as well as inducing NK cell proliferation. Furthermore, IL-15, either monomeric or as part of the TriKE, demonstrates the ability to reactivate latently HIV-infected T-cells isolated from infected patients in vitro.

A recent trial of an IL-15/IL-15Ra superagonist (Nant-803) in ART-treated HIV-infected patients also resulted in the detection of virus in the serum and immune activation. Together, these data indicate a role for an HIV-bnAb containing TriKE in the reactivation and elimination of the latently infected reservoir by harnessing NK cells ability to mediate ADCC.

Bi-specific fusions have been made that incorporate an anti-human anti-CD16 scFv derived from a human phage display library technology (McCall et al., 1999. Mol Immunol. 36:433-445). NK cells mediate antibody-dependent cell-mediated cytotoxicity (ADCC) through the CD16 (FcγRIII) receptor. Signaling through the CD16 receptor induces calcium fluxes and phosphorylation of ITAMs, triggering the release of lytic granules and cytokines such as interferon (IFNγ) and tumor necrosis factor (TNFα). A bi-specific molecule has been designed to trigger the CD16 receptor in conjunction with other targeting molecules (Gleason et al. Blood. 2014 (19):3016-26), a so-called bispecific killer engager (BiKE). With one scFv recognizing NK cells and a second scFv recognizing a tumor antigen, BiKEs can markedly enhance cytotoxic killing in various human cancers. One exemplary BiKE targeted CD33 and enhanced NK cell responses against acute myeloid leukemia (AML) and myelodyplastic syndrome (MDS). MDS is a clonal heterogeneous stem cell disorder characterized by normal or hypercellular bone marrow (BM) with peripheral blood (PB) cytopenias and an increased risk of progressing to AML.

NK cells are responsive to a variety of cytokines including, for example, IL-15, which is involved in NK cell homeostasis, proliferation, survival, activation, and/or development. IL-15 and IL-2 share several signaling components, including the IL-2/IL-15R□□ (CD122) and the common gamma chain (CD132). Unlike IL-2, IL-15 does not stimulate Tregs, allowing for NK cell activation while bypassing Treg inhibition of the immune response. Besides promoting NK cell homeostasis and proliferation, IL-15 can rescue NK cell functional defects that can occur in the post-transplant setting. IL-15 also can stimulate CD8+ T cell function, further enhancing its immunotherapeutic potential. In addition, based on pre-clinical studies, toxicity profiles of IL-15 may be more favorable than IL-2 at low doses.

IL-15 plays a role in NK cell development homeostasis, proliferation, survival, and activation. IL-15 and IL-2 share several signaling components including the IL-2/IL-15R□ (CD122) and the common gamma chain (CD132). IL-15 also activates NK cells, and can restore functional defects in engrafting NK cells after hematopoietic stem cell transplantation (HSCT).

This disclosure describes, in one aspect, tri-specific killer engager (TriKE) molecules that generally include one or more NK cell engager domains (e.g., CD16, CD16+CD2, CD16+DNAM, CD16+NKp46), one or more targeting domains (that target, e.g., a tumor cell or virally-infected cell), and one or more cytokine NK activating domains (e.g., IL-15, IL-12, IL-18, IL-21, or other NK cell enhancing cytokine, chemokine, and/or activating molecule), with each domain operably linked to the other domains. As used herein, the term “operably linked” refers to direct or indirect covalent linking. Thus, two domains that are operably linked may be directly covalently coupled to one another. Conversely, the two operably linked domains may be connected by mutual covalent linking to an intervening moiety (e.g., and flanking sequence). Two domains may be considered operably linked if, for example, they are separated by the third domain, with or without one or more intervening flanking sequences. In illustrative embodiments, the NK engaging domain is directed to CD16 and the NK activating domain is IL-15 or a functional fragment thereof.

The NK engaging domain can include any moiety that binds to and/or activates an NK cell and/or any moiety that blocks inhibition of an NK cell. In some embodiments, the NK engaging domain can include an antibody that selectively binds to a component of the surface of an NK cell. In other embodiments, the NK engaging domain can include a ligand or small molecule that selectively binds to a component of the surface of an NK cell. As used herein, the term “selectively binds” refers to the ability to differentiate between two or more alternatives such as, for example, having differential affinity, to any degree, for a particular target. As used herein, “antibody” refers generally an immunoglobulin or a fragment thereof and thus encompasses a monoclonal antibody, a fragment thereof (e.g., scFv, Fab, F(ab′)2, Fv or other modified forms), a combination of monoclonal antibodies and/or fragments thereof, and/or a combination of polyclonal antibodies. Thus, for brevity, reference to an antibody that selectively binds to a component of the surface of an NK cell includes any antibody fragment that exhibits the described binding character. Similarly, reference to a ligand that selectively binds to a component of the surface of an NK cell includes any fragment of the ligand that exhibits the described binding character.

In some embodiments, the NK engaging domain can selectively bind to a receptor at least partially located at the surface of an NK cell. In certain embodiments, the NK engaging domain can serve a function of binding an NK cell and thereby bring the NK into spatial proximity with a target to which the targeting domain—described in more detail below—selectively binds. In certain embodiments, however, the NK engaging domain can selectively bind to a receptor that activates the NK cell and, therefore, also possess an activating function. As described above, activation of the CD16 receptor can elicit antibody-dependent cell-mediated cytotoxicity. Thus, in certain embodiments, the NK engaging domain can include at least a portion of an anti-CD16 receptor antibody effective to selectively bind to the CD16 receptor. In other embodiments, the NK engager cell domain may interrupt mechanisms that inhibit NK cells.

One can design the NK engaging domain to possess a desired degree of NK selectivity and, therefore, a desired immune engaging character. For example, CD16 has been identified as Fc receptors FcγRIIIa (CD16a) and FcγRIIIb (CD16b). These receptors bind to the Fc portion of IgG antibodies that then activates the NK cell for antibody-dependent cell-mediated cytotoxicity. Anti-CD16 antibodies selectively bind to NK cells, but also can bind to neutrophils. Anti-CD16a antibodies selectively bind to NK cells, but do not bind to neutrophils. A TriKE embodiment that includes an NK engaging domain that includes an anti-CD16a antibody can bind to NK cells but not bind to neutrophils. Thus, in circumstances where one may want to engage NK cells but not engage neutrophils, one can design the NK engaging domain of the TriKE to include an anti-CD16a antibody.

While described herein in the context of various embodiments in which the NK engaging domain includes an anti-CD16 receptor scFv, the NK engaging domain can include any antibody or other ligand that selectively binds to the CD16 receptor. Moreover, the NK engaging domain can include an antibody or ligand that selectively binds to any NK cell receptor such as, for example, the cell cytotoxicity receptor 2B4, low affinity Fc receptor CD16, killer immunoglobulin like receptors (KIR), CD2, NKG2A, TIGIT, NKG2C, LIR-1, and/or DNAM-1.

The targeting domain can include any moiety that selectively binds to an intended target such as, for example, a tumor cell, a target in the cancer stroma, a target on an inhibitory cell such as myeloid derived suppressor cells that are CD33+, or a target on a virally-infected cell.

In other embodiments, the targeting domain can selectively bind to a target on a cell infected by a virus such as, for example, an adenovirus, HIV, CMV, and/or HPV. In illustrative examples herein, the targeting domain is an HIV epitope.

The NK activating domain can include an amino acid sequence that activates NK cells, promotes sustaining NK cells, or otherwise promotes NK cell activity. The NK activating domain can be, or can be derived from, one or more cytokines that can activate and/or sustain NK cells. As used herein, the term “derived from” refers to an amino acid fragment of a cytokine (e.g., IL-15) that is sufficient to provide NK cell activating and/or sustaining activity. In embodiments that include more than one NK activating domain, the NK activating domains may be provided in series or in any other combination. Additionally, each cytokine-based NK activating domain can include either the full amino acid sequence of the cytokine or may be an amino acid fragment, independent of the nature of other NK activating domains included in the

TriKE molecule. Exemplary cytokines on which an NK activating domain may be based include, for example, IL-15, IL-18, IL-12, and IL-21. Thus, while described in detail herein in the context of an exemplary model embodiment in which the NK activating domain is derived from IL-15, a TriKE may be designed using an NK activating domain that is, or is derived from, any suitable cytokine.

For brevity in this description, reference to an NK activating domain by identifying the cytokine on which it is based includes both the full amino acid sequence of the cytokine, any suitable amino acid fragment of the cytokine, and or a modified version of the cytokine that includes one or more amino acid substitutions. Thus, reference to an “IL-15” NK activating domain includes an NK activating domain that includes the full amino acid sequence of IL-15, an NK activating domain that includes a fragment of IL-15, or an NK activating domain such as, for example, IL-15N72D or IL-15N72A, that includes an amino acid substitution compared to the wild-type IL-15 amino acid sequence.

The use of an IL-15 NK activating domain in a TriKE can provide sustained NK cell activity—as evidenced in a mouse model showing human NK cells are dramatically elevated and cancer reduced—even after three weeks. NK cells are activated in mice to produce an array of anti-cancer factors and cytokines. An IL-15 NK activating domain somehow alters the chemistry of these molecules so that they refold more easily and/or are recoverable in greater yield, thus rendering the TriKE molecules more suitable for clinical scale-up.

In some embodiments, the molecule can further include a flanking sequence that can link two of the above-described domains. In some embodiments, the presence of the flanking sequence can further increase NK cell activation. One exemplary flanking sequence includes the 20 amino acids of SEQ ID NO:1 (see also US 2018/0282386). Another exemplary flanking sequence includes the seven amino acids of SEQ ID NO:2. Certain embodiments include more than one flanking sequence. As one example, SEQ ID NO:1 includes the flanking sequence of SEQ ID NO:3 to linked to the NK engaging domain (e.g., anti-CD16 receptor scFv) with the NK activating domain (e.g., IL-15). SEQ ID NO:1 also includes the flanking sequence of SEQ ID NO:4 to link the NK activating domain with the targeting domain (e.g., anti-CD33 scFv).

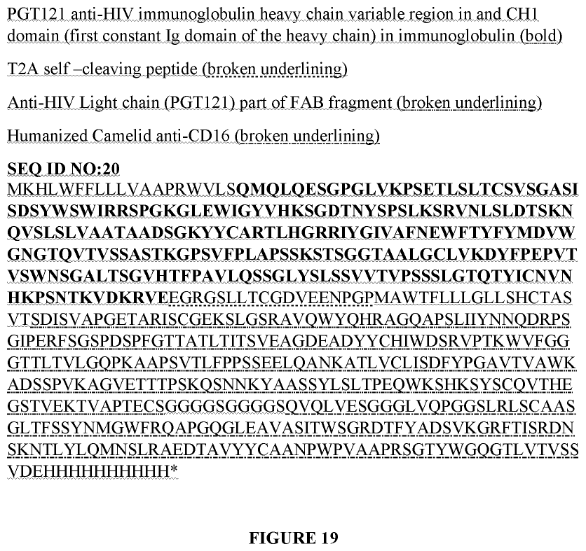

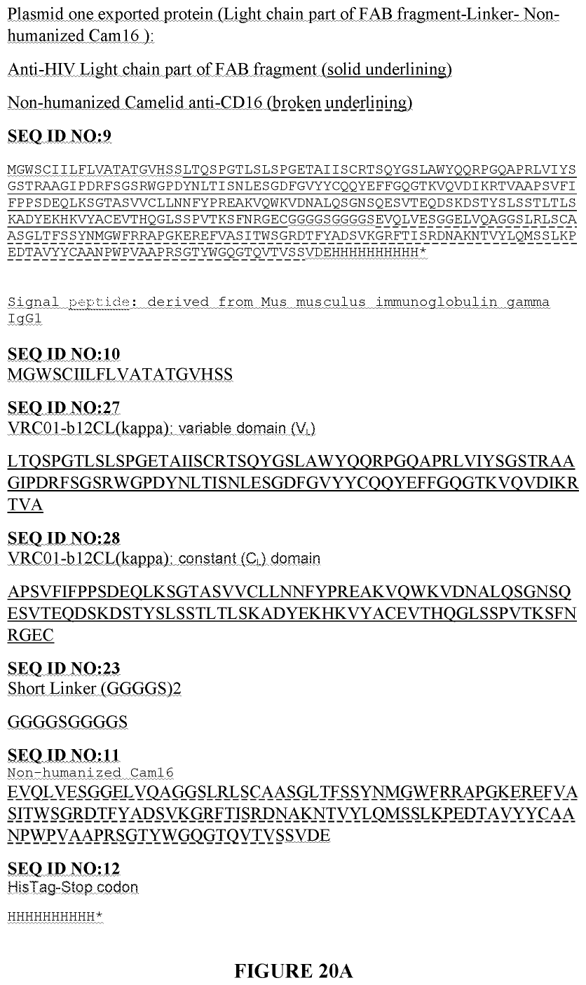

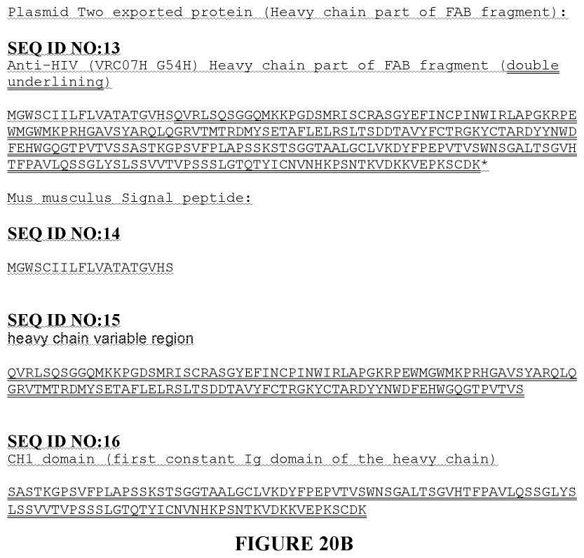

⁵¹Chromium release assays were performed with several different TriKEs to show that any scFv that targets cancer cells can be incorporated into a functional TriKE. Non-small cell lung cancer cells (NCI-H460) cells were incubated with the 1615EPCAM133 TriKE or the 1615NG2 TriKE. Both 1615NG2 and 1615EpCAM133 had activity at several different E:T ratios (20:1, 10:1, and 5:1). FIG. 19B shows melanoma cells were incubated with the 1615EPCAM133 TriKE. Mesothelin+EpCAM-NG2 MDA-435A melanoma cells were incubated with 1615EPCAM TriKE, or the 1615Meso TriKE (SEQ ID NO:32). Only 1615Meso had activity. Ovarian cancer cells (Ovcar3 cells) were incubated with the 1615NG2 TriKE or the 1615Meso TriKE. Both TriKEs induced NK cytolytic activity. Also, an anti-leukemic TriKE was made recognizing the leukemia markers CD19 and CD22. 16152219 TriKE was tested on CD22+CD19+ Raji cells and killed them very well (as well as rituximab). Together, these data show that any scFv can be inserted into the generalized TriKE structural platform of 1615X and the resulting TriKE can direct NK cells to respond to the scFv target and expand. Additional exemplary TriKE molecules are listed in Table 1.

TABLE 1
Exemplary TriKE molecules

| TriKE molecule | Target(s) | ADCC* | Expansion** | Activation*** |
|---|---|---|---|---|
| 161533 | CD33 | + | + | + |
| 1615EpCAM | EpCAM | + | + | + |
| 1615EpCAM133 | EpCAM/CD133 | +/+ | +/+ | +/+ |
| 1615133 | CD133 | + | + | + |
| 1615NG2 | NG2 | + | + | + |
| 1615Meso | mesothelin | + | + | + |
| 1615ROR-1 | ROR-1 | + | + | + |
| 16a1538 | CD38 | + | + | + |
| 1615IGF-1 | IGF1 | + | + | + |
| 1615Her2 | Her2/neu | + | + | + |
| 16152219 | CD22/CD19 | +/+ | +/+ | +/+ |
| Llama161533 | CD33 | + | + | + |
| 1615HIV | HIV | + | + | + |

*ADCC or cytotoxic activity enhanced over 30% by TriKE platform
**Expansion: TriKE enhances expansion of NK cells, BiKE does not.
***Activation: TriKE enhances the production of various anti-cancer cytokines including INFγ and TNFα.

In some embodiments, the NK cell engager can involve the use of a humanized CD16 engager derived from an animal nanobody. While an scFv has a heavy variable chain component and a light variable chain component joined by a linker, a nanobody consists of a single monomeric variable chain, e.g., a variable heavy chin or a variable light chain—that is capable of specifically engaging a target. A nanobody may be derived from an antibody of any suitable animal such as, for example, a camelid (e.g., a llama or camel) or a cartilaginous fish. A nanobody can provide superior physical stability, an ability to bind deep grooves, and increased production yields compared to larger antibody fragments.

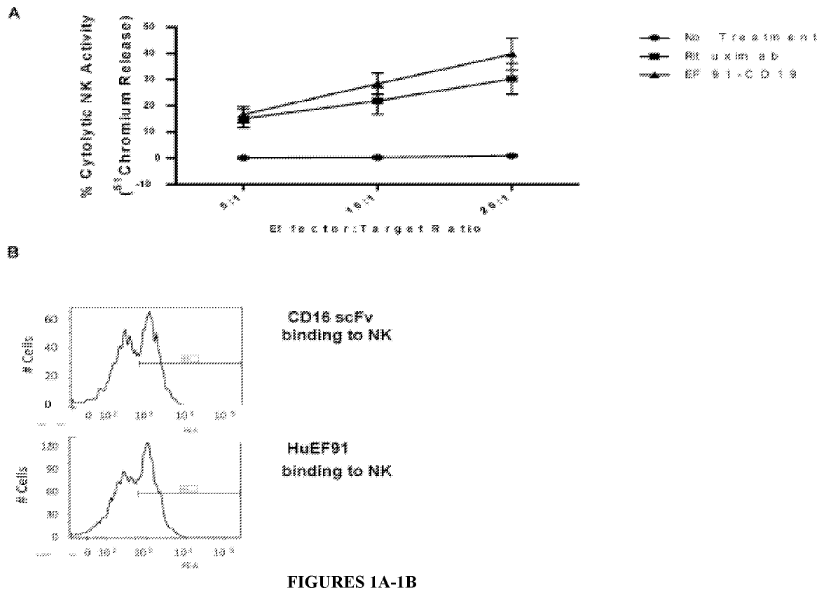

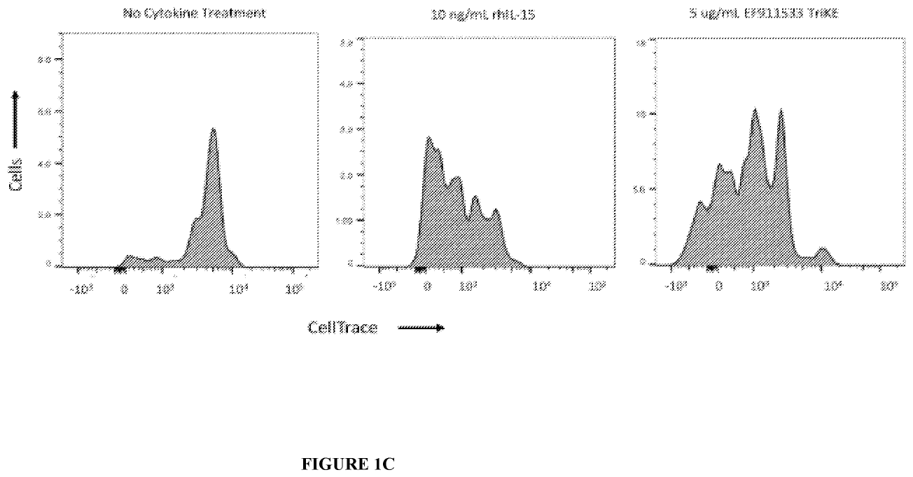

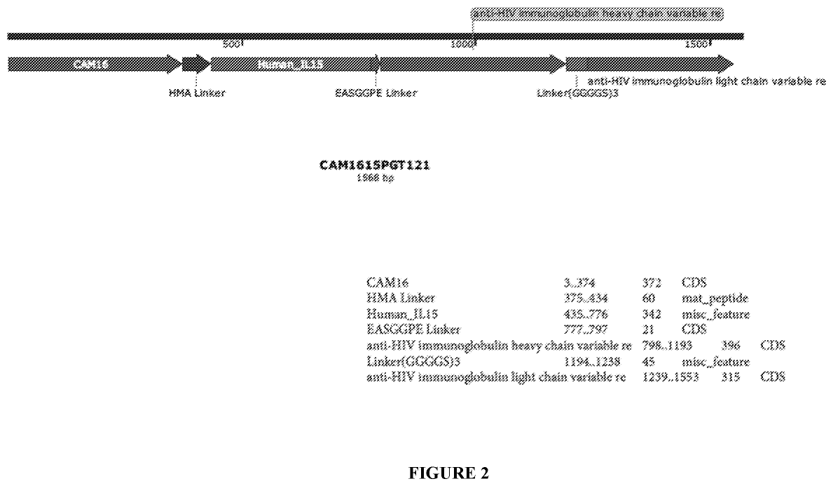

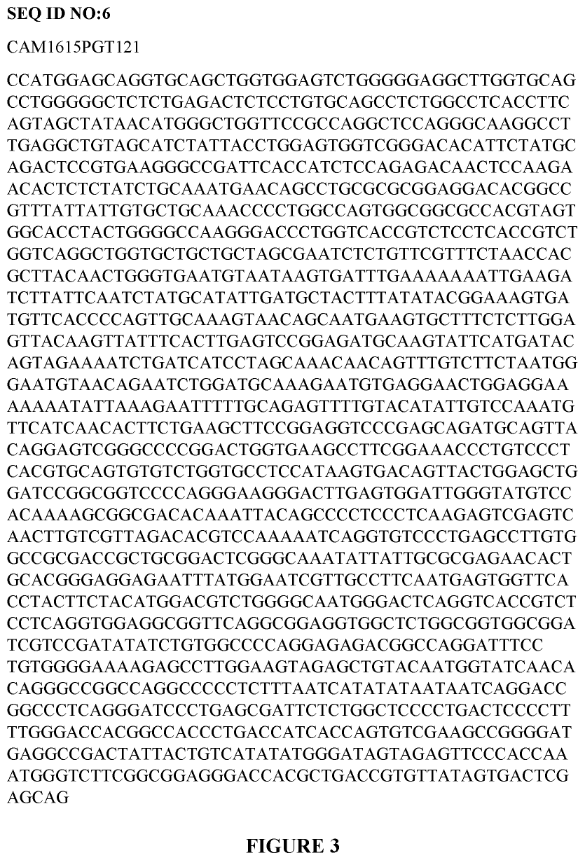

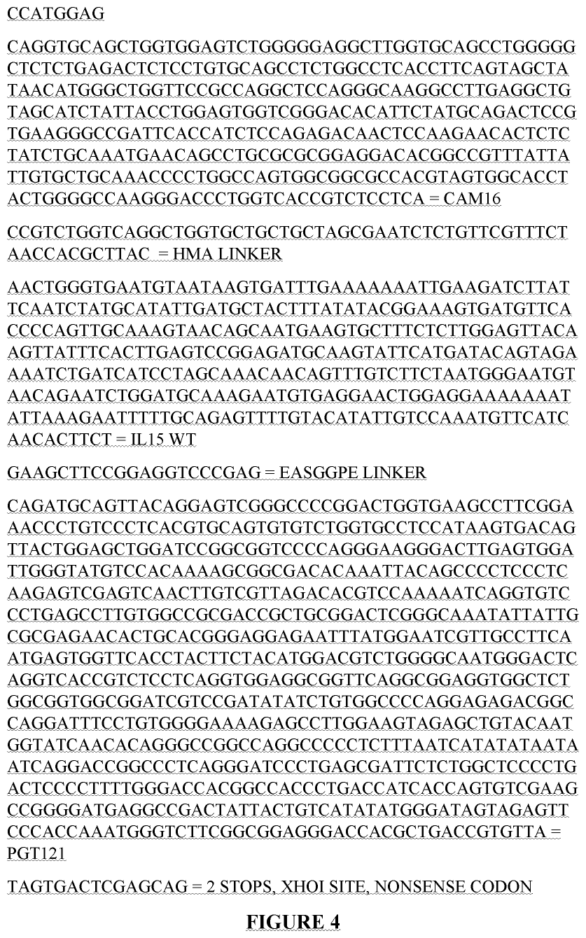

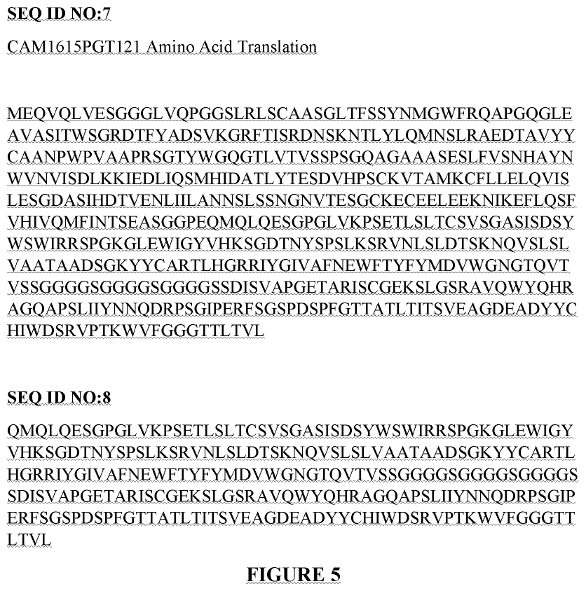

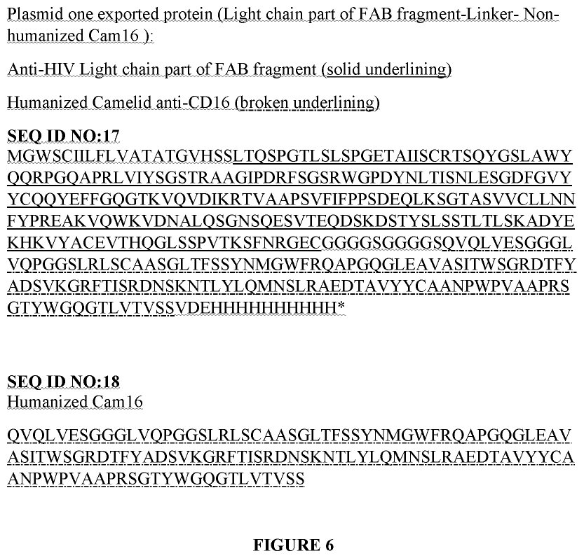

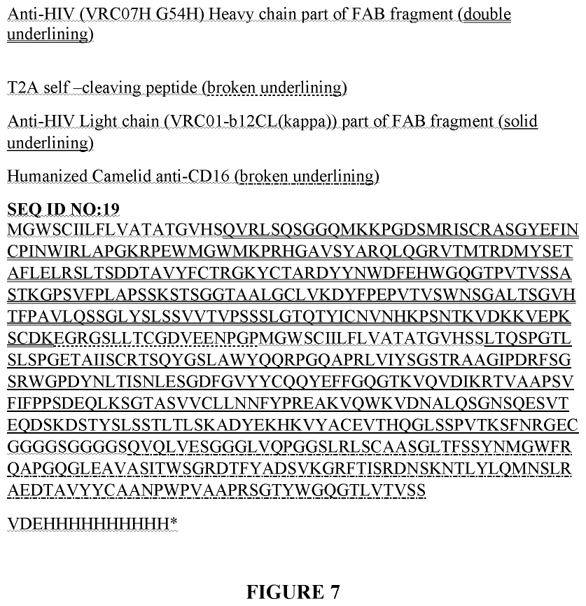

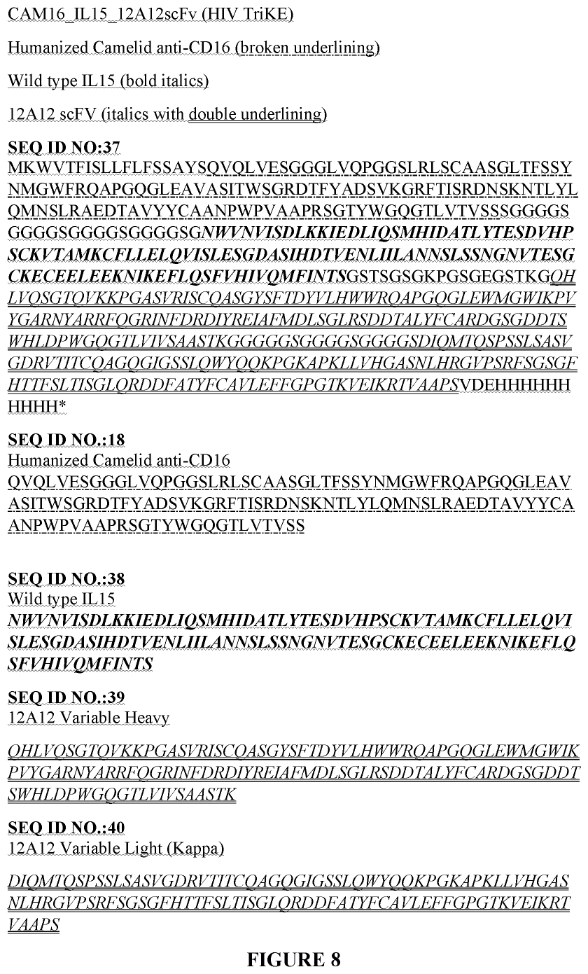

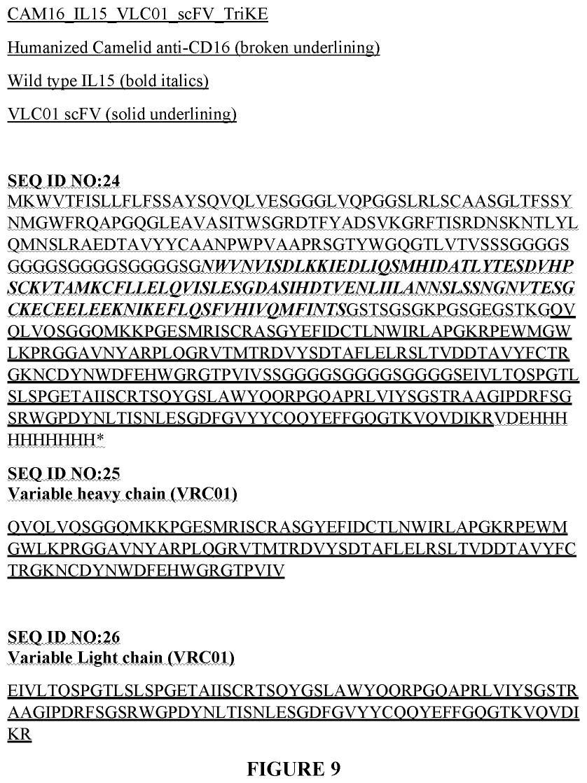

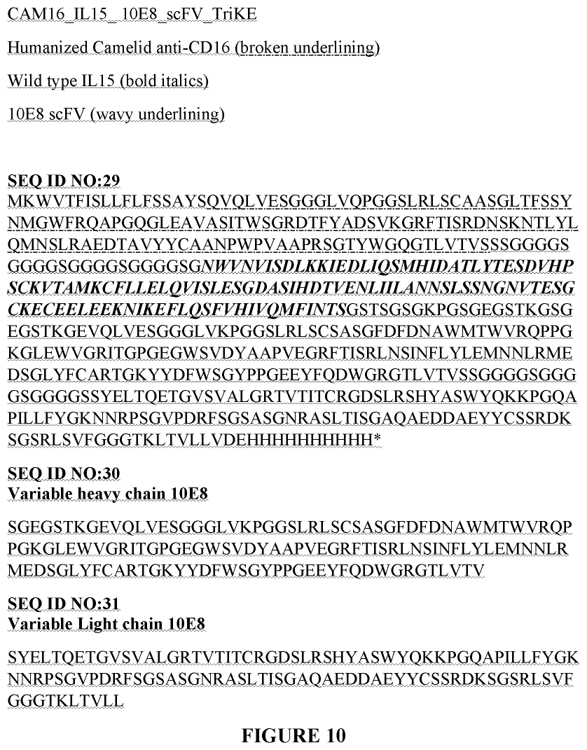

In one exemplary embodiment, a nanobody-based NK engager molecule can involve a humanized CD16 nanobody derived from a published llama nanobody (GeneBank sequence EF561291; Behar et al., 2008. Protein Eng Des Sel. 21(1):1-10), termed EF91. Llama EF91 was initially constructed into a BiKE containing CD19 to test the ability of this CD16 engager to drive NK cell activation. It showed functionality similar to rituximab-mediated killing in a chromium release assay with Raji targets (FIG. 1A). Upon confirming functionality of the molecule, the CDRs were cloned into a humanized camelid scaffold (Vincke et al., 2009. J Biol Chem. 284(5):3273-3284) to humanize the CD16 engager, now termed HuEF91. The binding of HuEF91 was equivalent to binding observed using a standard CD16 scFv, indicating that incorporating the llama nanobody variable heavy chain into the humanized backbone has not hindered the specificity of the molecule. The use HuEF91 as an NK engager in the TriKE molecules described herein can increase drug yield, increase stability, and/or increase NK-cell-mediated ADCC efficacy.

In some embodiments, an immune engager as described herein can be used to stimulate a patient's own immune system to eliminate tumor cells. Although studies show that T cells, genetically modified to express chimeric antigen receptors (CARs), are powerful clinical mediators of anti-tumor activity, production of T-CARs is costly and complex. Other disadvantages include the risk of cytokine toxicity and long-term persistence of T-CARs resulting in interaction with healthy tissue or neoplastic transformation. As described herein, a tri-specific killer engager can serve as a mediator of ADCC and can expand NK cells without the need of extracorporal genetic modification and gene therapy, providing a potential advantage over the T-CAR system. Because the immune engager is rapidly cleared, the response cannot be indefinitely sustained, perhaps reducing the risk of cytokine toxicity of the immune engagers compared to T-CARs.

In some embodiments, a tri-specific killer engager includes a cytokine. In some embodiments, a tri-specific killer engager preferably includes IL-15. IL-15 does not induce Tregs and IL-15 is a regulator of NK cells. In addition to improving activation and cytotoxicity, IL-15 can regulate and initiate anti-apoptotic and proliferative signals on NK cells, leading to enhanced NK cell expansion and survival. These characteristics can be beneficial during the use of the tri-specific killer engager in the treatment against cancer. In some embodiments, including IL-15 in the tri-specific killer engager can mediate directed delivery of the TriKE to the NK/Target cell synapse, potentially causing IL-15 to accumulate at a tumor site more effectively than systemic IL-15.

In some embodiments, the immune engager increases the secretion of an immune cell-mediated cytokine. In some embodiments, the cytokine secretion is preferably antigen specific. In some embodiments, this cytokine can include IFN-γ, GM-CSF, IL-6, IL-8, and/or TNF-α. In some embodiments, this cytokine production is preferably at physiologic levels. In some embodiments, this cytokine production is at a level lower than the level observed in an IL-12/IL-18 stimulated NK cell (Papadakis et al., 2004. J Immunol. 172:7002-7007). As shown in Example 2, measuring hallmark inflammatory cytokines including GM-CSF, IL-6, IL-8, TNF-α using a cytokine Luminex analysis demonstrates a statistically significant difference in GM-C SF secretion between BiKE and TriKE but no difference in the secretion of other cytokines.

In some embodiments, the immune engager increases proliferation of a lymphocyte. The lymphocyte can include, for example, an NK cell, a γδ-T cell, and/or, a CD8 T cell. One can design a TetraKE, or larger molecule, that includes more than one NK cell engager domain and/or more than one NK activating domain.

In another aspect, this disclosure describes methods of killing a target cell in a subject. Generally, the method includes administering to the subject a TriKE molecule in an amount effective to induce NK-mediated killing of the target cells. “Treat” or variations thereof refer to reducing, limiting progression, ameliorating, or resolving, to any extent, the symptoms or signs related to a condition. As used herein, “ameliorate” refers to any reduction in the extent, severity, frequency, and/or likelihood of a symptom or clinical sign characteristic of a particular condition; “symptom” refers to any subjective evidence of disease or of a patient's condition; and “sign” or “clinical sign” refers to an objective physical finding relating to a particular condition capable of being found by one other than the patient.

A “treatment” may be therapeutic or prophylactic. “Therapeutic” and variations thereof refer to a treatment that ameliorates one or more existing symptoms or clinical signs associated with a condition. “Prophylactic” and variations thereof refer to a treatment that limits, to any extent, the development and/or appearance of a symptom or clinical sign of a condition. Generally, a “therapeutic” treatment is initiated after the condition manifests in a subject, while “prophylactic” treatment is initiated before a condition manifests in a subject. Thus, in certain embodiments, the method can involve prophylactic treatment of a subject at risk of developing a condition. “At risk” refers to a subject that may or may not actually possess the described risk. Thus, for example, a subject “at risk” for developing a specified condition is a subject that possesses one or more indicia of increased risk of having, or developing, the specified condition compared to individuals who lack the one or more indicia, regardless of the whether the subject manifests any symptom or clinical sign of having or developing the condition. Exemplary indicia of a condition can include, for example, genetic predisposition, ancestry, age, sex, geographical location, lifestyle, or medical history. Treatment may also be continued after symptoms have resolved, for example to prevent or delay their recurrence.

In the case of a subject infected with HIV, for example, “treatment” may include a reduction in viral load and/or an amelioration of symptoms.

In some cases, the treatment can involve administering the TriKE molecule to a subject so that the TriKE molecule can stimulate endogenous NK cells in vivo. Using a TriKE molecule as a part of an in vivo can make NK cells antigen specific with simultaneous co-stimulation, enhancement of survival, and expansion, which may be antigen specific. In other cases, the TriKE can be used in vitro as an adjuvant to NK cell adoptive transfer therapy.

Accordingly, a TriKE molecule, whether an NK-activating TriKE or a T-cell-activating TriKE, may be administered before, during, or after the subject first exhibits a symptom or clinical sign of the condition. Treatment initiated before the subject first exhibits a symptom or clinical sign associated with the condition may result in decreasing the likelihood that the subject experiences clinical evidence of the condition compared to a subject to which the TriKE molecule is not administered, decreasing the severity of symptoms and/or clinical signs of the condition, and/or completely resolving the condition. Treatment initiated after the subject first exhibits a symptom or clinical sign associated with the condition may result in decreasing the severity of symptoms and/or clinical signs of the condition compared to a subject to which the composition is not administered, and/or completely resolving the condition.

The TriKE molecule can be any embodiment of the TriKE molecule described above having a targeting domain that selectively binds to an appropriate target cell population. In some cases, the target cell can include a virus infected cell so that the method can involve treating the viral infection. Thus, in some embodiments, the method can include ameliorating at least one symptom or clinical sign of the viral infection.

In various embodiments, the TriKE targeting domain can include a polypeptide that selectively binds to, for example, mesothelin or a viral antigen on HIV for example.

As used herein, a “subject” can be any animal such as, for example, a mammal (e.g., human, dog, cat, horse, cow, sheep, goat, monkey, etc.). In certain embodiments, the subject can be a human.

A TriKE molecule described herein may be formulated with a pharmaceutically acceptable carrier. As used herein, “carrier” includes any solvent, dispersion medium, vehicle, coating, diluent, antibacterial, and/or antifungal agent, isotonic agent, absorption delaying agent, buffer, carrier solution, suspension, colloid, and the like. The use of such media and/or agents for pharmaceutical active substances is well known in the art. Except insofar as any conventional media or agent is incompatible with the active ingredient, its use in the therapeutic compositions is contemplated. Supplementary active ingredients also can be incorporated into the compositions. As used herein, “pharmaceutically acceptable” refers to a material that is not biologically or otherwise undesirable, i.e., the material may be administered to an individual along with a TriKE molecule without causing any undesirable biological effects or interacting in a deleterious manner with any of the other components of the pharmaceutical composition in which it is contained.

A TriKE molecule may therefore be formulated into a pharmaceutical composition. The pharmaceutical composition may be formulated in a variety of forms adapted to a preferred route of administration. Thus, a composition can be administered via known routes including, for example, oral, parenteral (e.g., intradermal, transcutaneous, subcutaneous, intramuscular, intravenous, intraperitoneal, etc.), or topical (e.g., intranasal, intrapulmonary, intramammary, intravaginal, intrauterine, intradermal, transcutaneous, rectally, etc.). A pharmaceutical composition can be administered to a mucosal surface, such as by administration to, for example, the nasal or respiratory mucosa (e.g., by spray or aerosol). A composition also can be administered via a sustained or delayed release.

Thus, a TriKE molecule may be provided in any suitable form including but not limited to a solution, a suspension, an emulsion, a spray, an aerosol, or any form of mixture. The composition may be delivered in formulation with any pharmaceutically acceptable excipient, carrier, or vehicle. For example, the formulation may be delivered in a conventional topical dosage form such as, for example, a cream, an ointment, an aerosol formulation, a non-aerosol spray, a gel, a lotion, and the like. The formulation may further include one or more additives including such as, for example, an adjuvant, a skin penetration enhancer, a colorant, a fragrance, a flavoring, a moisturizer, a thickener, and the like.

A formulation may be conveniently presented in unit dosage form and may be prepared by methods well known in the art of pharmacy. Methods of preparing a composition with a pharmaceutically acceptable carrier include the step of bringing a TriKE molecule into association with a carrier that constitutes one or more accessory ingredients. In general, a formulation may be prepared by uniformly and/or intimately bringing the active molecule into association with a liquid carrier, a finely divided solid carrier, or both, and then, if necessary, shaping the product into the desired formulations.

The amount of TriKE molecule administered can vary depending on various factors including, but not limited to, the specific TriKE molecule being used, the weight, physical condition, and/or age of the subject, and/or the route of administration. Thus, the absolute weight of TriKE molecule included in a given unit dosage form can vary widely, and depends upon factors such as the species, age, weight and physical condition of the subject, and/or the method of administration. Accordingly, it is not practical to set forth generally the amount that constitutes an amount of TriKE molecule effective for all possible applications. Those of ordinary skill in the art, however, can readily determine the appropriate amount with due consideration of such factors.

In some embodiments, the method can include administering sufficient TriKE molecule to provide a dose of, for example, from about 100 ng/kg to about 50 mg/kg to the subject, although in some embodiments the methods may be performed by administering TriKE molecule in a dose outside this range. In some of these embodiments, the method includes administering sufficient TriKE molecule to provide a dose of from about 10 μg/kg to about 5 mg/kg to the subject, for example, a dose of from about 100 μg/kg to about 1 mg/kg.

Alternatively, the dose may be calculated using actual body weight obtained just prior to the beginning of a treatment course. For the dosages calculated in this way, body surface area (m2) is calculated prior to the beginning of the treatment course using the Dubois method: m2=(wt kg0.425×height cm0.725)×0.007184.

In some embodiments, the method can include administering sufficient TriKE molecule to provide a dose of, for example, from about 0.01 mg/m2 to about 10 mg/m2.

In some embodiments, a TriKE molecule may be administered, for example, from a single dose to multiple doses per week, although in some embodiments the method can be performed by administering a TriKE molecule at a frequency outside this range. In certain embodiments, a TriKE molecule may be administered from about once per month to about five times per week.

In some embodiments, the method further includes administering one or more additional therapeutic agents. The one or more additional therapeutic agents may be administered before, after, and/or coincident to the administration of a TriKE molecule. A TriKE molecule and the additional therapeutic agents may be co-administered. As used herein, “co-administered” refers to two or more components of a combination administered so that the therapeutic or prophylactic effects of the combination can be greater than the therapeutic or prophylactic effects of either component administered alone. Two components may be co-administered simultaneously or sequentially. Simultaneously co-administered components may be provided in one or more pharmaceutical compositions. Sequential co-administration of two or more components includes cases in which the components are administered so that each component can be present at the treatment site at the same time. Alternatively, sequential co-administration of two components can include cases in which at least one component has been cleared from a treatment site, but at least one cellular effect of administering the component (e.g., cytokine production, activation of a certain cell population, etc.) persists at the treatment site until one or more additional components are administered to the treatment site. Thus, a co-administered combination can, in certain circumstances, include components that never exist in a chemical mixture with one another. In other embodiments, the TriKE molecule and the additional therapeutic agent may be administered as part of a mixture or cocktail. In some aspects, the administration of TriKE molecule may allow for the effectiveness of a lower dosage of other therapeutic modalities when compared to the administration of the other therapeutic agent or agents alone, thereby decreasing the likelihood, severity, and/or extent of the toxicity observed when a higher dose of the other therapeutic agent or agents is administered.

In some embodiments, of the method can include administering sufficient TriKE molecule as described herein and administering the at least one additional therapeutic agent demonstrates therapeutic synergy. In some aspects of the methods of the present invention, a measurement of response to treatment observed after administering both a TriKE molecule as described herein and the additional therapeutic agent is improved over the same measurement of response to treatment observed after administering either the TriKE molecule or the additional therapeutic agent alone. In some embodiments, an additional therapeutic agent can include an additional agent that targets HIV, for example atazanavir (Reyataz); darunavir (Prezista); fosamprenavir (Lexiva); lopinavir; ritonavir (Norvir); tipranavir (Aptivus) or acyclovir. Other antivirals are known to those of skill in the art and can be combined with an TriKe composition before, during or following TriKe administration.

In the preceding description and following claims, the term “and/or” means one or all of the listed elements or a combination of any two or more of the listed elements; the terms “comprises,” “comprising,” and variations thereof are to be construed as open ended—i.e., additional elements or steps are optional and may or may not be present; unless otherwise specified, “a,” “an,” “the,” and “at least one” are used interchangeably and mean one or more than one; and the recitations of numerical ranges by endpoints include all numbers subsumed within that range (e.g., 1 to 5 includes 1, 1.5, 2, 2.75, 3, 3.80, 4, 5, etc.).

In the preceding description, particular embodiments may be described in isolation for clarity. Unless otherwise expressly specified that the features of a particular embodiment are incompatible with the features of another embodiment, certain embodiments can include a combination of compatible features described herein in connection with one or more embodiments.

For any method disclosed herein that includes discrete steps, the steps may be conducted in any feasible order. And, as appropriate, any combination of two or more steps may be conducted simultaneously.

The present invention is illustrated by the following examples. It is to be understood that the particular examples, materials, amounts, and procedures are to be interpreted broadly in accordance with the scope and spirit of the invention as set forth herein.

### EXAMPLE 1

### Construction of 1615 AntiHIV

Since 1615x is a platform technology, it is also possible to use anti-viral scFvs that are or are not associated with cancer development. Synthesis and assembly of a hybrid polynucleotide encoding the TriKE 1615antiHIV (SEQ ID NO:5) was accomplished using DNA shuffling and ligation techniques. The fully-assembled polynucleotide has, from the 5′ end to the 3′ end, an Ncol restriction site; an ATG initiation codon; the VH and VL regions of the anti-CD16 scFv, a 20 amino acid segment (PSGQAGAAASESLFVSNHAY SEQ ID NO:1), modified IL-15, a seven amino acid linker (EASGGPE SEQ ID NO:2), and an anti-HIV scFv; and finally a XhoI restriction site.

An HIV-Envelope specific, IL-15 containing Tri-specific killer engager (TriKE) both reactivates and directs NK cell killing towards HIV-infected T-cells.

While advancements in efficacy and use of anti-retroviral drugs have substantially ameliorated the health and longevity of HIV-infected individuals, these drugs are merely a stop-gap to prevent progression to AIDS and to limit further transmission of the virus. Despite the use of antiretrovirals to suppress HIV replication, infected individuals retain reservoirs of latently HIV-infected cells that, upon cessation of anti-retroviral therapy, could reactivate and re-establish an active infection. A curative solution necessitates the reactivation and subsequent destruction of these latently infected cells. The antibody response to HIV infection, while present, is generally ineffective due to the high rate of mutation of the virus which can rapidly eliminate epitopes recognized by the generated antibodies. However, in recent years a variety of HIV-specific antibodies have been identified in infected individuals which have strong neutralizing effects but a poor ability to elicit antibody dependent cell-mediated cytotoxicity (ADCC). Thus, we have designed bi- and tri-specific killer engagers (BiKE and TriKE) composed of a short-chain variable fragment derived from a broadly-neutralizing antibody (bnAb) against HIV-Env and a CD16 engager linked by an IL-15 molecule. The purpose of this tri-specific antibody construct is to utilize the broad specificity of these antibodies to target HIV while redirecting NK cell killing specifically to actively replicating infected cells though its recognition of membrane expressed Env and triggering NK cell degranulation though the low affinity Fc receptor, CD16. The addition of IL-15 as a linker should further activate NK cells thereby enhancing their response. IL-15 has also been identified as a potential reactivator of latently infected cells. Initial studies from our lab show enhanced NK cell cytokine production and killing of infected targets expressing HIV-Env when incubated with the HIV-specific constructs. PBMC from healthy donors incubated with the TriKE showed marked increases in immune cell activation in NK, CD4 and CD8 subsets, as well as inducing NK cell proliferation. Furthermore, IL-15, either monomeric or as part of the TriKE, demonstrates the ability to reactivate latently HIV-infected T-cells isolated from infected patients in vitro. A recent trial of an IL-15/IL-15Ra superagonist (Nant-803) in ART-treated HIV-infected patients also resulted in the detection of virus in the serum and immune activation. Together, these data indicate a potential role for an HIV-bnAb containing TriKE in the reactivation and elimination of the latently infected reservoir by harnessing NK cells ability to mediate ADCC. As further examples, a broadly neutralizing antibody (bnAb) shown in Table 2 below can be used in the compositions and methods described herein in a 16/15/X TriKE. Antibodies shown in Table 2 can be used in TriKE constructs comprising camCD16 /IL-15/ anti-HIV bnAb, for example. Further examples of databases listing HIV blocking antibodies useful in the present invention can be found publicly at the following sites and are hereby incorporated by reference in their entirety:

https://web.archive.org/web/20131230231821/http://bnaber.org/;
https://www.hiv.lanl.gov/content/immunology/ab_search;
https://web.archive.org/web/20131230231821/http://bnaber.org/

TABLE 2
Broadly Neutralizing HIV Antibodies

| Broadly Neutralizing | |
|---|---|
| HIV Antibodies- | Patent/Application^{#} |
| VRC01, 02, 03, and 07 | U.S. Pat. No. 9,175,070 |
| | U.S. Pat. No. 8,637,036 |
| | U.S. Pat. No. 9,738,703 |
| | U.S. Pat. No. 10,035,845 |
| | U.S. Pat. No. 9,695,230 |
| | U.S. Pat. No. 10,035,844 |
| | U.S. application Ser. No. 13/498,125 |
| | U.S. application Ser. No. 13/429,286 |
| | U.S. application Ser. No. 14/864,705 |
| | U.S. application Ser. No. 15/661,867 |
| | U.S. application Ser. No. 61/385,531 |
| | U.S. application Ser. No. 13/429,279 |
| | U.S. application Ser. No. 61/568,520 |
| | U.S. application Ser. No. 61/613,431 |
| | U.S. application Ser. No. 61/698,452 |
| | PCT Application No. PCT/US2012/068827 |
| | U.S. application Ser. No. |
| | 14/363,740 US |
| | Application No. |
| | 15/612,846 US |
| | Application No. |
| | 16/044,083 |
| N6 | U.S. application Ser. No. 15/559,791 |
| | U.S. application Ser. No. 62/136,228 |
| | U.S. application Ser. No. 62/250,378 |
| | PCT Application No. PCT/US2016/023145 |
| 10E8 | U.S. application Ser. No. 62/250,360 |
| | PCT Application No. PCT/US2016/060390 |
| | U.S. application Ser. No. 15/772,443 |
| | CA Application No. 3003878 |
| | CN Application No. 201680077520.X |
| | EP Application No. 16801639.2 |
| | IN Application No. 201837016184 |
| | ZA Application No. 2018/02875 |
| | AU Application No. 2016349392 |
| | U.S. Pat. No. 9,475,862 |
| | U.S. application Ser. No. 15/226,744 |
| | U.S. application Ser. No. 61/672,708 |
| | U.S. application Ser. No. 61/698,480 |
| | PCT Application No. PCT/US2012/063958 |
| | U.S. application Ser. No. 14/356,557 |
| | U.S. application Ser. No. 15/699,902 |
| | U.S. application Ser. No. 61/638,437 |
| | PCT Application No. PCT/US2013/038214 |
| PGT121 | U.S. Pat. No. 10,239,935 |

^{#}The disclosures of patents and applications listed above are incorporated herein by reference, specifically with respect to the bnAb sequences.

### EXAMPLE 2

### A Tri-Specific Killer Engager (Trike) Against Mesothelin Targets NK Cells Towards Lung Cancer

NK cells are important effectors in the treatment of hematological malignancies, but have so far been less effective in the treatment of solid tumors. Lung cancer cells are generally refractory to NK cell killing, but we wanted to determine whether a small molecule that redirects lysis of NK cells against the common tumor antigen, mesothelin, could enhance NK cell killing in the lung cancer setting. Mesothelin is a surface protein that is overexpressed in a number of cancers, including the aggressive cancer of the lung lining: mesothelioma.

A comparison of peripheral blood NK cells from healthy donors and newly diagnosed cancer patients revealed that lung cancer patients maintained expression of CD16 (the Fc receptor) at the cell surface with no differences in the major subsets of NK cells. We therefore designed a tri-specific killer engager (TriKE) consisting of a single domain antibody (sdAb) against CD16 and a single chain variable fragment (scFv) against mesothelin. The sdAb and scFv were linked together by recombinant IL-15. When tested on peripheral blood NK cells from healthy donors, this drug was capable of enhancing NK cell proliferation in vitro. In addition, when peripheral blood NK cells were cultured with 9 different lung cancer lines, the TriKE increased degranulation (>60% in some cases) and IFNγ production (>30% in some cases), specifically against the cancer cells.

NK cells from the peripheral blood of lung cancer patients also proliferated in response to the drug alone. Moreover, when treated with the TriKE, patients' cells increased degranulation (>60%) and IFNγ production (>40%, which was significantly more than healthy donor responses) in response to lung cancer cells.

Checkpoint blocking antibodies are the current standard of care for lung cancer patients and our further investigations are focusing on the combination of this mesothelin-targeted TriKE with checkpoint blockade, both in vitro and in vivo.

### EXAMPLE 3

### Transient Expression of Anti-HIV/CAM16 Bikes and Trikes

Experimental Design

For the VRC01-b12CL/VRCO7H G54H (see Table 4 for bnAbs), we started by first testing a VRC Fab based BiKE. bNAbs take years to evolve and accumulate three times as many mutations as other antibodies.

Expression Constructs

For expression in mammalian cell lines the VRC01-b12CL (light chain) was cloned into suitable expression vector (pCoof40), along with a short linker (GGGGS2) and the non-humanized CAM16. The cloning method used was Gibson assembly or the related method HiFi assembly (New England Biolabs). The humanized CAM16 BiKE was also constructed the same way. The VRCO7H G54H plasmid was not modified. A large-scale preparation of both plasmid DNAs was produced by ZymoPURE Plasmid Maxiprep Kit (Zymo Research).

BiKE Protein Expression

We used a suspension-adapted CHO or HEK293 cell line (ExpiCHO /Expi293F) for high levels of transient protein expression. A 1:1 ratio of heavy-to-light chain containing plasmids (1 uG DNA/mL of ExpiCHO Media) was co-transfected using ExpiFectamine CHO or 293 Transfection Kits (Thermo Fisher) and incubated with shaking in a temperature and CO2 controlled incubator according to manufacturer's instructions. 4-5 days later, or when viability was measured below 75% viable cells, the supernatant was harvested by centrifugation at 2000 RPM in a benchtop centrifuge followed by filtration through a 0.22 micron filter. The filtered supernatant was then stored at -80° C.

When ready for purification the drug was thawed and isolated using TALON metal affinity resin according to manufacturer's instructions. Drug was subsequently quantified using the Bradford assay with BSA used as the standard. Purity was accessed using densitometry on a BoltTM 4-12% Bis-Tris plus SDS-Page gel run in BoltTM IVIES SDS running buffer and stained with Coomassie dye G-250.

### EXAMPLE 4

### HIV-Specific Bikes and Trikes

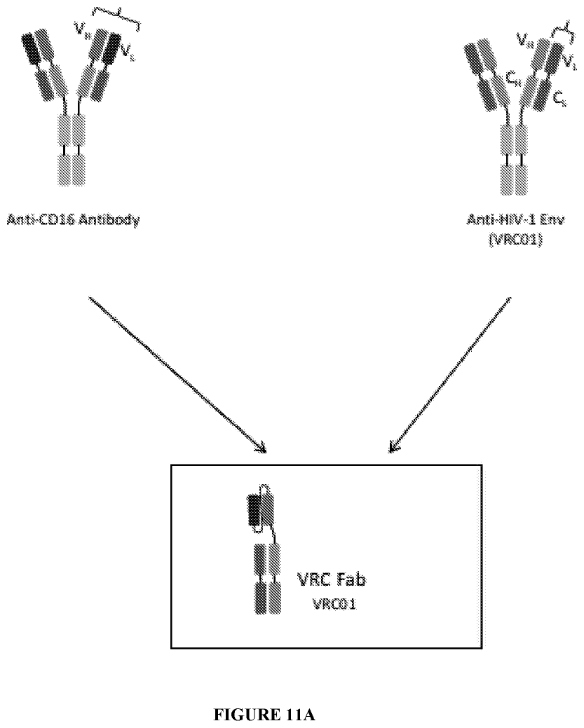

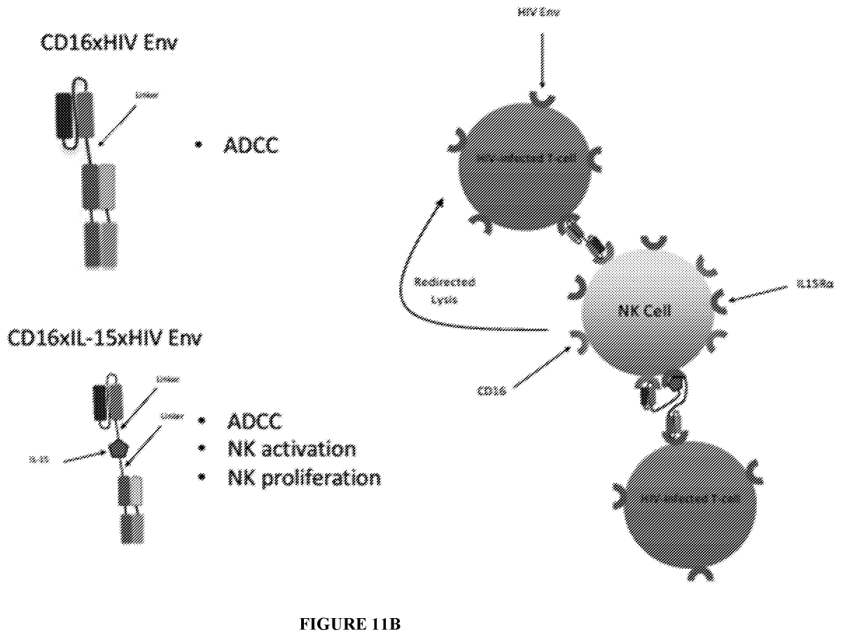

FIG. 11 shows the structure and proposed function of HIV-specific BiKEs and TriKEs. FIG. 11(A) Shown is a schematic illustrating the origin of of the components for the initial bi-specific HIV-targeting construct comprised of an anti-CD16 short-chain variable fragment linked to a Fab derived from the HIV broadly neutralizing antibody (bnAb) VRC01. FIG. 11(B) Schematic and proposed function of an HIV bi- and tri-specific killer engager (BiKE and TriKE, respectively). The BiKE binds HIV-envelope expressed on infected cells though the bnAb component while the anti-CD16 moiety binds the NK cell and signals through CD16, eliciting a functional response. Similar activity is proposed using a TriKE with the addition of the IL-15 linkers ability to activate the NK cell, enhancing its function and inducing a proliferative response. The IL-15 component may also activate latently infected T-cells thereby making them susceptible to recognition by the HIV-TriKE and the immune system at large.

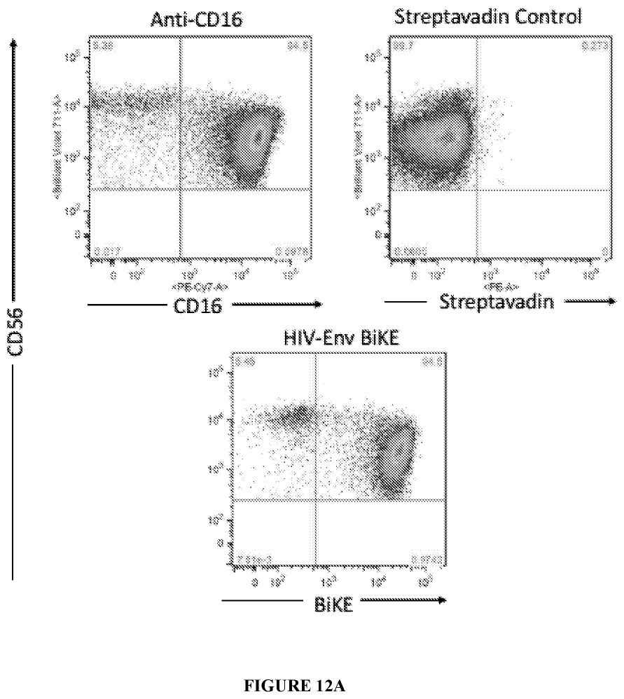

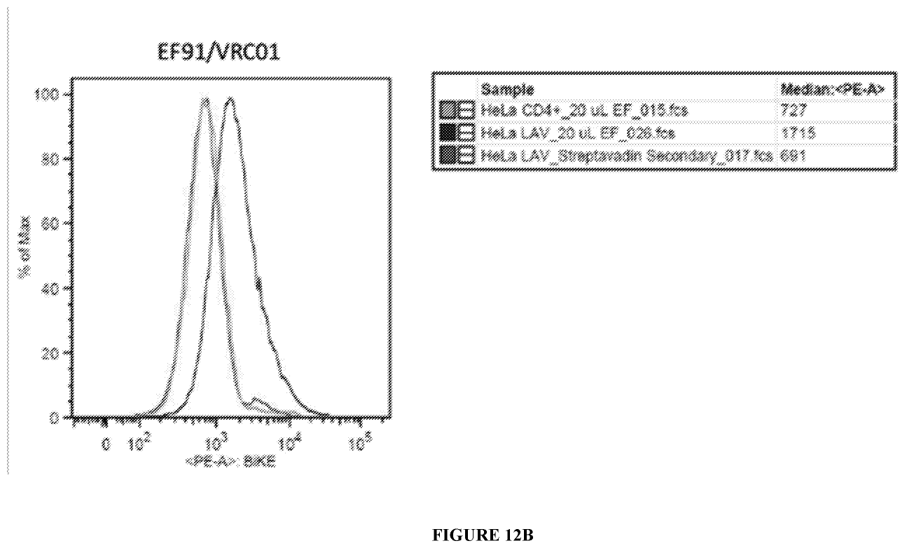

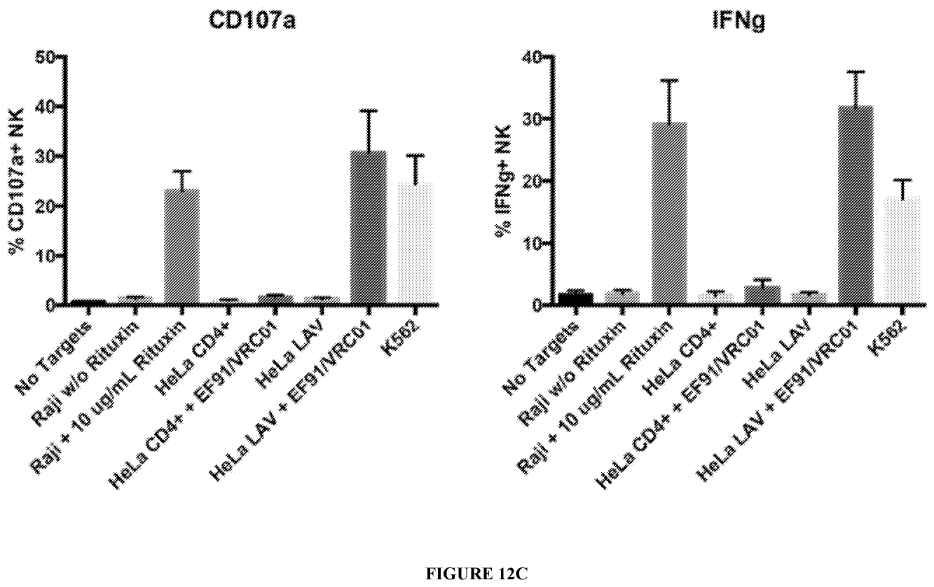

FIG. 12 shows that a HIV-Env specific BiKE binds CD16-expressing NK cells, HIV-infected cell lines, and induces and HIV-specific NK cell response. FIG. 12 (A) Healthy donor purified peripheral blood NK cells were stained for CD16, streptavidin control or His-tagged BiKE with biotinylated anti-His and fluorochrome conjugated streptavidin. The BiKE binds NK cells reflective of CD16 expression. FIG. 12(B) Uninfected CD4-expressing HeLa cells or HeLa-CD4 infected with HIV were stained with HIV-Env BiKE. The BiKE specifically bound the infected HeLa-CD4 but not the uninfected HeLa-CD4 demonstrating specificity of the BiKE for cells expressing HIV envelope. FIG. 12(C) Purified healthy donor NK cells were incubated with infected or uninfected HeLa-CD4 cells with and without HIV-Env BiKE. K562 cells and Rajis with Rituxin were used as controls. HIV-Env BiKE elicited a strong degranulation and cytokine response only against the infected HeLa cells, demonstrating the specific activity of the BiKE. These responses were equivalent to the Raji+Rituxin control.

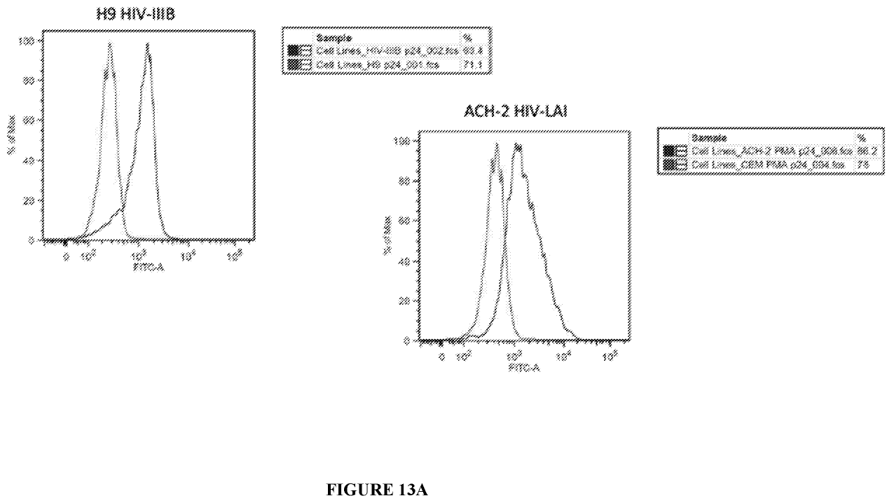

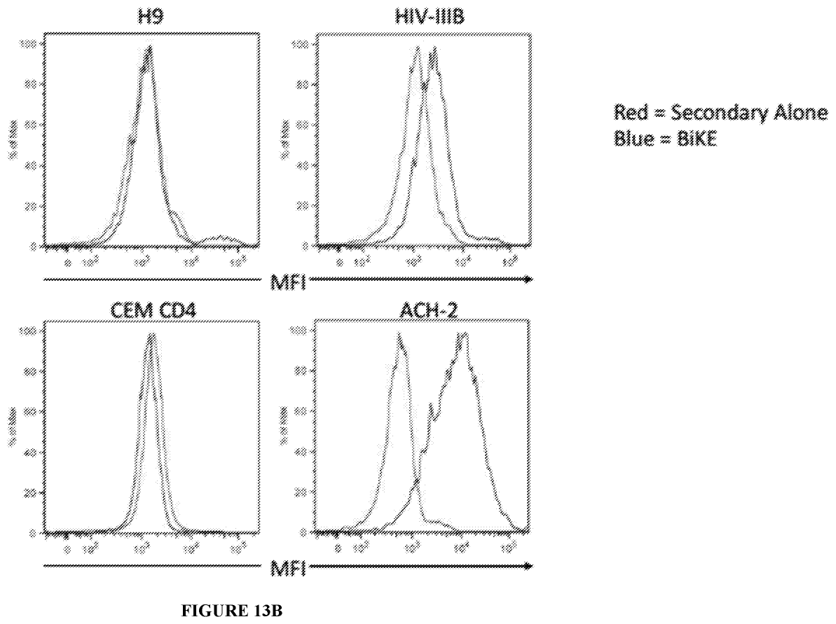

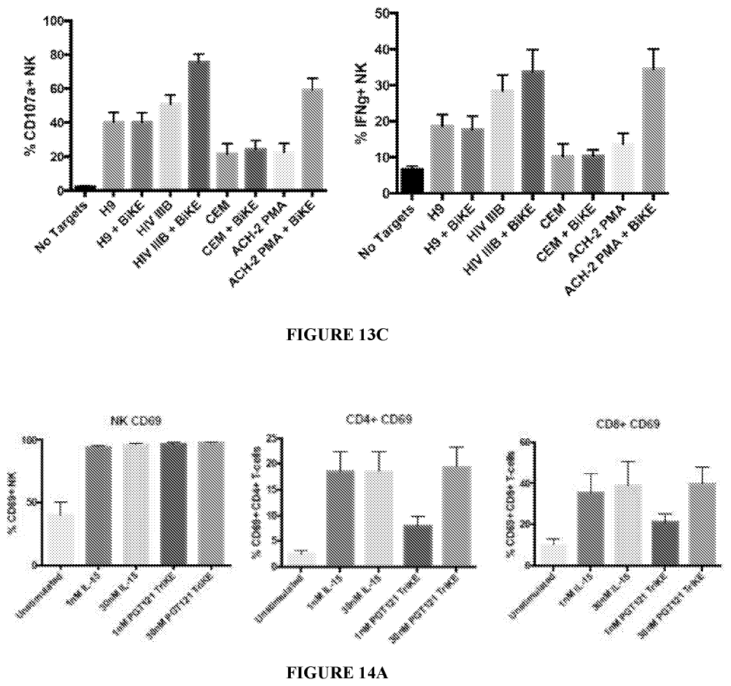

FIG. 13 shows that an HIV-Env BiKE specifically binds primary infected T-cell lines and mediates NK cell killing. FIG. 13(A) Two HIV-infected T-cell lines, H9 HIV-IBB and ACH-2, or their uninfected counterparts, H9 and CEM CD4, were intracellularly stained for HIV capsid protein to confirm active HIV replication. FIG. 13(B) The same infected and uninfected T-cell lines were stained with the His-tagged HIV-Env BiKE and biotinylated anti-His+ streptavidin or the secondary alone. BiKE showed no binding to the uninfected T-cell lines but bound both infected clones demonstrating a specificity for actively infected T-cells. FIG. 13(C) Purified NK cells from healthy donors were co-cultured with uninfected or HIV-infected T-cell lines with and without HIV-Env BiKE and assessed for NK degranulation (CD107a) and IFNg production. The HIV-BiKE enhanced both NK cell degranulation and cytokine production specifically against the infected T-cell lines but not the uninfected.

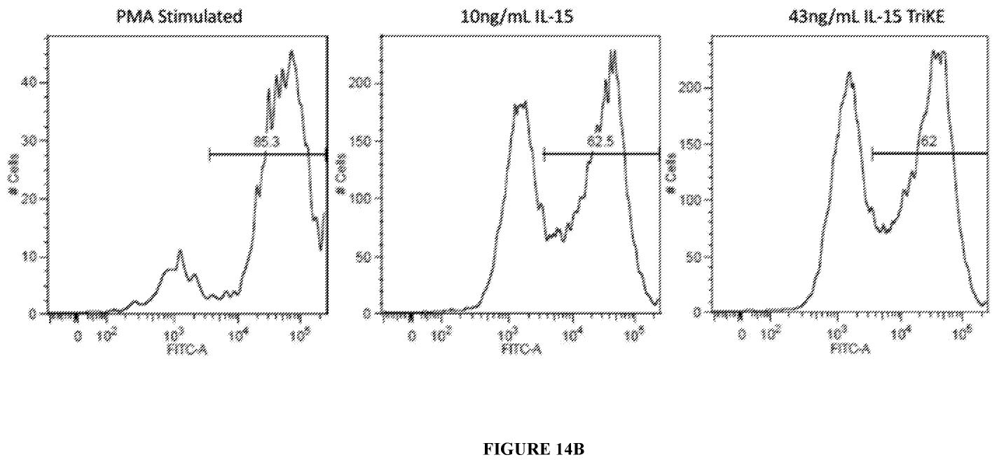

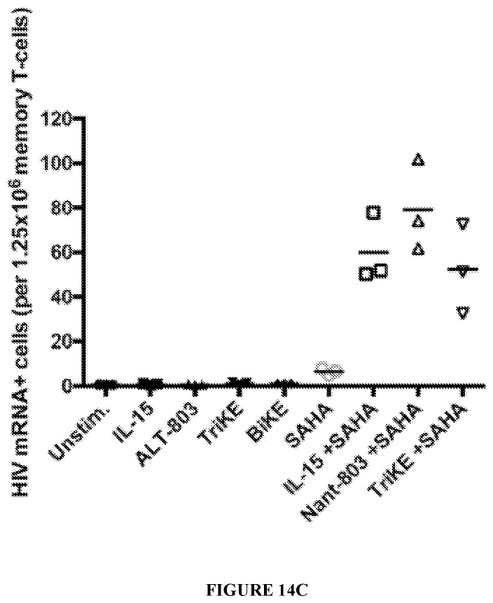

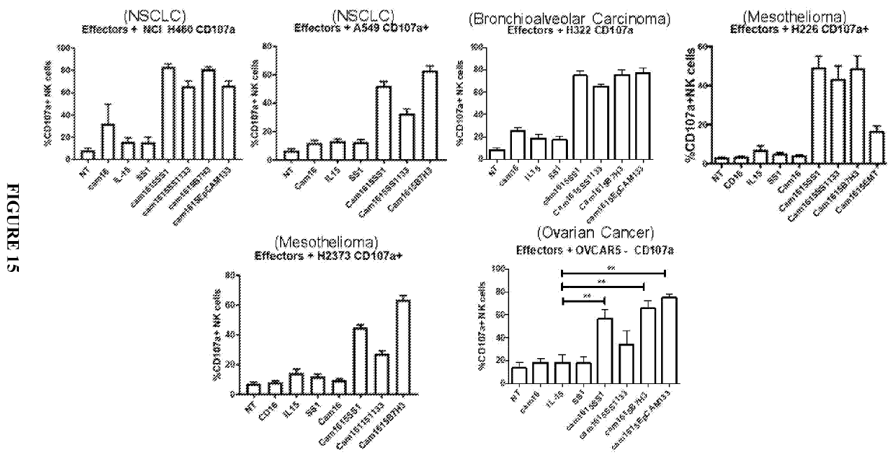

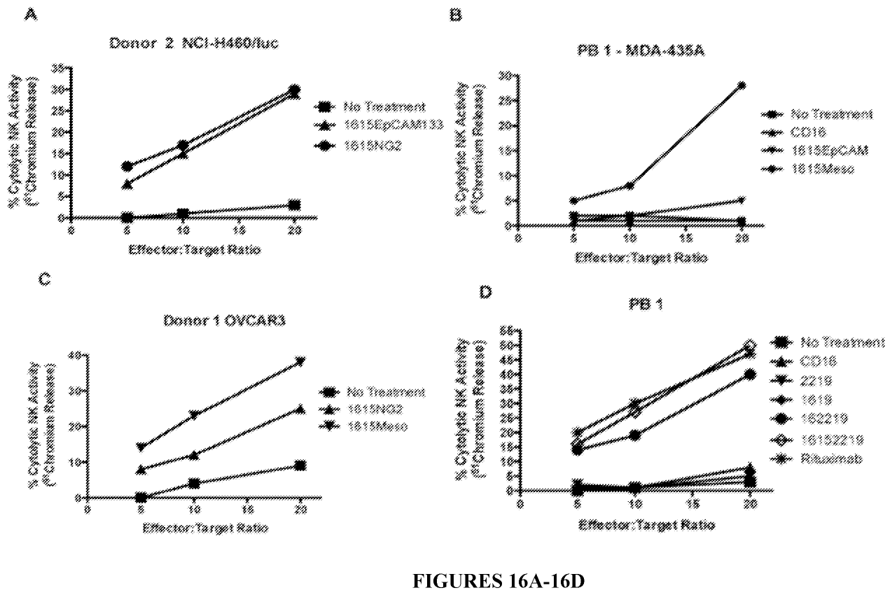

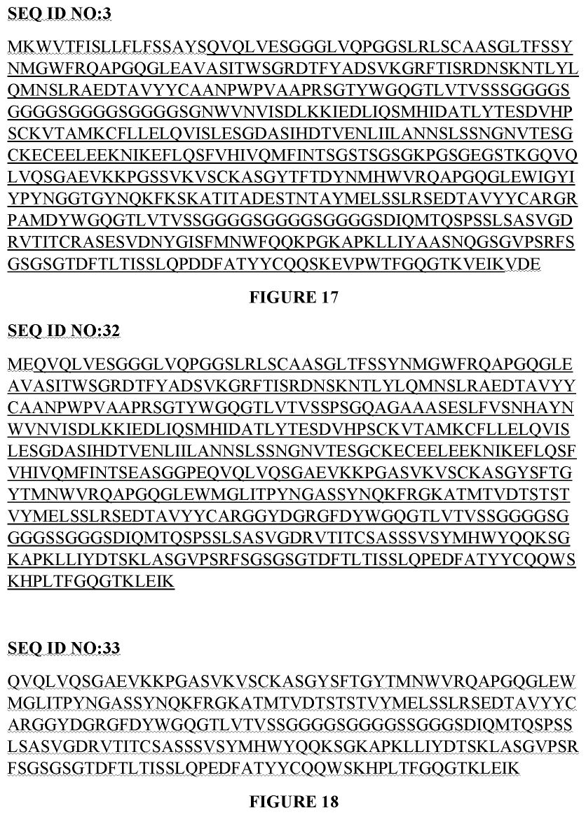

FIG. 14 shows that an IL-15 containing HIV-TriKE activates immune subsets and induces viral transcription in latently infected primary and T-cell lines. (A) Peripheral blood mononuclear cells were incubated with equimolar rhlL-15 or IL-15 containing HIV-TriKE for 16 hours. NK and T-cell subsets were evaluated by flow cytometry for activation by CD69 expression. FIG. 14 (B) The latently infected human CD4+ T-cell line, ACH-2, was incubated for 48 hours in the presence of 10 nM PMA, 10 ng/mL rhIL-15 or equimolar IL-15 containing TriKE. Cells were then washed and intracellularly stained for HIV-gag (p24). Both IL-15 alone and TriKE induced significant viral reactivation as shown by p24 expression. FIG. 14 (C) Purified CD4+ memory T-cells were isolated from anti-retroviral treated, HIV-infected patients and cultured with rhIL-15, the IL-15 Superagonist, Nant-803, or IL-15 containing TriKE. Each condition was incubated with or without the HDAC inhibitor, SAHA for 72 hours. Cells were then harvested and a nested PCR reaction was done to identify HIV mRNA.

Both BiKE and TriKE molecules can be generated using the above methods that include cotransfection of two plasmids or polynucleotides that separately encode light and heavy chains, for example. Two proteins can also be produced from a single plasmid or polynucleotide using a 2A self-cleaving peptide or an IRES, for example. Exemplary BiKE and TriKE molecules and amino acid sequences are shown in the Figures and sequences herein).

In addition to the various embodiments described in the specification above, the following additional embodiments are contemplated herein.

Embodiment 1. A compound comprising:
an NK engaging domain comprising a moiety that selectively binds to CD16;
an NK activating domain operably linked to the NK engaging domain comprising IL-15 or a functional fragment thereof and a targeting domain that selectively binds to a viral antigen and is operably linked to the NK activating domain and the NK engaging domain.

Embodiment 2. The compound of claim 1, wherein the CD16 comprises CD16a.

Embodiment 3. The compound of claim 1, wherein the viral antigen is present on an infected cell.

Embodiment 4. The compound of claim 1, wherein the viral antigen is derived from HIV, CMV, HPV, HCV, or an adenovirus.

Embodiment 5. The compound of claim 1, wherein the viral antigen is derived from HIV.

Embodiment 6. The compound of claim 1, wherein the NK engaging domain moiety comprises an antibody or a binding fragment thereof or a nanobody.

Embodiment 7. The compound of claim 6, wherein the antibody fragment comprises an scFv, a F(ab)2, or a Fab.

Embodiment 8. The compound of claim 6, wherein the antibody or a binding fragment thereof or the nanobody is human, humanized, or camelid.

Embodiment 9. The compound of claim 6, wherein the antibody or a binding fragment thereof or the nanobody is camelid.

Embodiment 10. The compound of claim 6, wherein the IL-15 comprises an amino acid sequence of SEQ ID NO: 4 or a functional variant thereof.

Embodiment 11. The compound of claim 10, wherein the functional variant of IL-15 comprises an N72D or N72A amino acid substitution as compared to SEQ ID NO:4.

Embodiment 12. The compound of claim 1, wherein the targeting domain moiety comprises an antibody or a binding fragment thereof or a nanobody.

Embodiment 13. The compound of claim 12, wherein the antibody binding fragment comprises an scFv, a F(ab)2, or a Fab.

Embodiment 14. The compound of claim 1, wherein the NK engaging domain comprises CD16, the NK activating domain comprises IL-15, and the targeting domain selectively binds to a viral antigen derived from HIV.

Embodiment 15. The compound of claim 1, wherein the NK engaging domain comprises CD16a, the NK activating domain comprises IL-15, and the targeting domain selectively binds to a viral antigen derived from HIV.

Embodiment 16. The compound of claim 1, wherein the NK engaging domain comprises NKG2c, the NK activating domain comprises IL-15, and the targeting domain selectively binds to a viral antigen derived from HIV.

Embodiment 17. The compound of claim 1, comprising at least one flanking sequence linking two of the domains.

Embodiment 18. The compound of claim 17, further comprising a second flanking sequence linking the two linked domains with the third domain.

Embodiment 19. The compound of claim 18, wherein the flanking sequences flank the NK activating domain.

Embodiment 20. The compound of claim 18, wherein a first flanking sequence is C-terminal to the NK engaging domain and wherein a second flanking sequence is N-terminal to the anti-viral targeting domain.

Embodiment 21. The compound of claim 1, further comprising a second targeting domain.

Embodiment 22. The compound of claim 1, further comprising a second NK engaging domain.

Embodiment 23. The compound of claim 1, further comprising a second NK activating domain.

Embodiment 24. The compound of claim 1, wherein the compound is SEQ ID NO:5, 7, 24, 29 or 37.

Embodiment 25. A composition comprising:
the compound of any of claims 1-24; and
a pharmaceutically acceptable carrier.

Embodiment 26. A method comprising:
administering to a subject the compound of any of claims 1-25 in an amount effective to induce NK-mediated killing of a target cell.

Embodiment 27. The method of claim 26, wherein the target cell is infected with a virus.

Embodiment 28. The method of claim 27, wherein the virus is HIV, CMV, HPV, HCV, or an adenovirus.

Embodiment 29. The method of claim 28, wherein the virus is HIV.

Embodiment A method for stimulating expansion of NK cells in vivo, the method comprising:

administering to a subject an amount of the compound of any of claims 1-25 effective to stimulate expansion of NK cells in the subject.

Embodiment 31. The method of claim 30, wherein the subject is infected with a virus.

Embodiment 32. The method of claim 31, wherein the virus is HIV, CMV, HPV, HCV, or an adenovirus.

Embodiment 33. The method of claim 32, wherein the virus is HIV.

Embodiment 34. A method of treating viral infection in a subject, the method comprising:

administering to the subject an amount of the compound of any of claims 1-25 effective for treating the viral infection.

Embodiment 35. The method of claim 34, wherein the subject is infected with HIV, CMV, HPV, HCV, or an adenovirus.

Embodiment 36. The method of claim 35, wherein the subject is infected with HIV.

Embodiment 37. An isolated nucleic acid sequence of SEQ ID NO:6.

Embodiment 38. An isolated amino acid sequence of SEQ ID NO:7.

Embodiment 39. An isolated amino acid sequence comprising the sequence of camCD16/IL-15/SEQ ID NO:8.

Embodiment 40. An isolated amino acid sequence comprising SEQ ID NO:9, 17, 27, 28, 13, 15, 16, 17, 18, 19, 20.

Embodiment 41. The isolated amino acid of claim 40, further comprising an isolated amino acid sequence of SEQ ID NO:10.

Embodiment 42. An isolated amino acid sequence comprising SEQ ID NO:18 operably linked to IL-15.

Embodiment 43. An isolated amino acid sequence of SEQ ID NO:20-26.

Embodiment 44. A method of making the compound of any of claims 1-24 comprising:
(i) co-transfecting into mammalian cells a first polynucleotide comprising a nucleotide sequence encoding an amino acid sequence comprising an immunoglobulin heavy chain of SEQ ID NO:22, 25, 30 or 39 and a second polynucleotide comprising a nucleotide sequence encoding an amino acid sequence comprising an immunoglobulin light chain of SEQ ID NO:21, 26, 31 or 40, respectively; and
(ii) collecting a supernatant from the mammalian cells, wherein the resulting compound binds to a viral antigen.

Embodiment 45. The method of claim 44, wherein the viral antigen is derived from HIV.

Embodiment 46. The method of claim 45, wherein the viral antigen is Env.

Embodiment 47. An isolated DNA sequence encoding the amino acid sequences of SEQ ID NOs:21, 22, 25, 26, 30, 31, 39 or 40.

Embodiment 48. A pharmaceutical composition comprising SEQ ID NO:7, 24, 29, 32, 34, 36 and 37 in a pharmaceutically acceptable carrier.

Embodiment 49. A method of treating a subject comprising administering to the subject a pharmaceutical composition comprising SEQ ID NO:5, 7, 24, 29, 32, 34, 36 and 37 in a pharmaceutically acceptable carrier.

Embodiment 50. A method of treating a subject having or being at risk for developing AIDS, comprising administering to the subject a pharmaceutical composition comprising SEQ ID NO:5, 7, 24, 29, and 37.

The complete disclosure of all patents, patent applications, and publications, and electronically available material cited herein are incorporated by reference in their entirety. Although the invention has been described with reference to the above examples, it will be understood that modifications and variations are encompassed within the spirit and scope of the invention. Accordingly, the invention is limited only by the following claims.

SEQUENCES
Linker
SEQ ID NO: 1
PSGQAGAAASESLFVSNHAY
Linker
SEQ ID NO: 2
EASGGPE
Llamal61533
SEQ ID NO: 3
MKWVTFISLLFLFSSAYSQVQLVESGGGLVQPGGSLRLSC
AASGLTFSSYNMGWFRQAPGQGLEAVASITWSGRDTFYAD
SVKGRFTISRDNSKNTLYLQMNSLRAEDTAVYYCAANPWP
VAAPRSGTYWGQGTLVTVSSSGGGGSGGGGSGGGGSGGGG
SGNWVNVISDLKKIEDLIQSMHIDATLYTESDVHPSCKVT
AMKCFLLELQVISLESGDASIHDTVENLIILANNSLSSNG
NVTESGCKECEELEEKNIKEFLQSFVHIVQMFINTSGSTS
GSGKPGSGEGSTKGQVQLVQSGAEVKKPGSSVKVSCKASG
YTFTDYNMHWVRQAPGQGLEWIGYIYPYNGGTGYNQKFKS
KATITADESTNTAYMELSSLRSEDTAVYYCARGRPAMDYW
GQGTLVTVSSGGGGSGGGGSGGGGSDIQMTQSPSSLSASV
GDRVTITCRASESVDNYGISFMNWFQQKPGKAPKLLIYAA
SNQGSGVPSRFSGSGSGTDFTLTISSLQPDDFATYYCQQS
KEVPWTFGQGTKVEIKVDE
Il-15 (human)
SEQ ID NO: 4
NWVNVISDLKKIEDLIQSMHIDATLYTESDVHPSCKVTAM
KCFLLELQVISLESGDASIHDTVENLIILANNSLSSNGNV
TESGCKECEELEEKNIKEFLQSFVHIVQMFINTS
1615antiHIV
SEQ ID NO: 5
MEVQLVESGGGVVRPGGSLRLSCAASGFTFDDYGMSWVRQ
APGKGLEWVSGINWNGGSTGYADSVKGRFTISRDNAKNSL
YLQMNSLRAEDTAVYYCARGRSLLFDYWGQGTLVTVSRGG
GGSGGGGSGGGGSSELTQDPAVSVALGQTVRITCQGDSLR
SYYASWYQQKPGQAPVLVIYGKNNRPSGIPDRFSGSSSGN
TASLTITGAQAEDEADYYCNSRDSSGNHVVFGGGTKLTVL
PSGQAGAAASESLFVSNHAYNWVNVISDLKKIEDLIQSMH
IDATLYTESDVHPSCKVTAMKCFLLELQVISLESGDASIH
DTVENLIILANDSLSSNGNVTESGCKECEELEEKNIKEFL
QSFVHIVQMFINTSEASGGPEMGWSCIILFLVATATGVHS
QVRLSQSGGQMKKPGDSMRISCRASGYEFINCPINWIRLA
PGKRPEWMGWMKPRHGAVSYARQLQGRVTMTRDMYSETAF
LELRSLTSDDTAVYFCTRGKYCTARDYYNWDFEHWGQGTP
VTVSSASTKGPSVFPLAPSSKSTSGGTAALGCLVKDYFPE
PVTVSWNSGALTSGVHTFPAVLQSSGLYSLSSVVTVPSSS
LGTQTYICNVNHKPSNTKVDKKVEPKSCDK
Cam1615PGT121
SEQ ID NO: 6
CCATGGAGCAGGTGCAGCTGGTGGAGTCTGGGGGAGGCTT
GGTGCAGCCTGGGGGCTCTCTGAGACTCTCCTGTGCAGCC
TCTGGCCTCACCTTCAGTAGCTATAACATGGGCTGGTTCC
GCCAGGCTCCAGGGCAAGGCCTTGAGGCTGTAGCATCTAT
TACCTGGAGTGGTCGGGACACATTCTATGCAGACTCCGTG
AAGGGCCGATTCACCATCTCCAGAGACAACTCCAAGAACA
CTCTCTATCTGCAAATGAACAGCCTGCGCGCGGAGGACAC
GGCCGTTTATTATTGTGCTGCAAACCCCTGGCCAGTGGCG
GCGCCACGTAGTGGCACCTACTGGGGCCAAGGGACCCTGG
TCACCGTCTCCTCACCGTCTGGTCAGGCTGGTGCTGCTGC
TAGCGAATCTCTGTTCGTTTCTAACCACGCTTACAACTGG
GTGAATGTAATAAGTGATTTGAAAAAAATTGAAGATCTTA
TTCAATCTATGCATATTGATGCTACTTTATATACGGAAAG
TGATGTTCACCCCAGTTGCAAAGTAACAGCAATGAAGTGC
TTTCTCTTGGAGTTACAAGTTATTTCACTTGAGTCCGGAG
ATGCAAGTATTCATGATACAGTAGAAAATCTGATCATCCT
AGCAAACAACAGTTTGTCTTCTAATGGGAATGTAACAGAA
TCTGGATGCAAAGAATGTGAGGAACTGGAGGAAAAAAATA
TTAAAGAATTTTTGCAGAGTTTTGTACATATTGTCCAAAT
GTTCATCAACACTTCTGAAGCTTCCGGAGGTCCCGAGCAG
ATGCAGTTACAGGAGTCGGGCCCCGGACTGGTGAAGCCTT
CGGAAACCCTGTCCCTCACGTGCAGTGTGTCTGGTGCCTC
CATAAGTGACAGTTACTGGAGCTGGATCCGGCGGTCCCCA
GGGAAGGGACTTGAGTGGATTGGGTATGTCCACAAAAGCG
GCGACACAAATTACAGCCCCTCCCTCAAGAGTCGAGTCAA
CTTGTCGTTAGACACGTCCAAAAATCAGGTGTCCCTGAGC
CTTGTGGCCGCGACCGCTGCGGACTCGGGCAAATATTATT
GCGCGAGAACACTGCACGGGAGGAGAATTTATGGAATCGT
TGCCTTCAATGAGTGGTTCACCTACTTCTACATGGACGTC
TGGGGCAATGGGACTCAGGTCACCGTCTCCTCAGGTGGAG
GCGGTTCAGGCGGAGGTGGCTCTGGCGGTGGCGGATCGTC
CGATATATCTGTGGCCCCAGGAGAGACGGCCAGGATTTCC
TGTGGGGAAAAGAGCCTTGGAAGTAGAGCTGTACAATGGT
ATCAACACAGGGCCGGCCAGGCCCCCTCTTTAATCATATA
TAATAATCAGGACCGGCCCTCAGGGATCCCTGAGCGATTC
TCTGGCTCCCCTGACTCCCCTTTTGGGACCACGGCCACCC
TGACCATCACCAGTGTCGAAGCCGGGGATGAGGCCGACTA
TTACTGTCATATATGGGATAGTAGAGTTCCCACCAAATGG
GTCTTCGGCGGAGGGACCACGCTGACCGTGTTATAGTGAG
TCGAGGAG
Cam1615PGT121
SEQ ID NO: 7
MEQVQLVESGGGLVQPGGSLRLSCAASGLTFSSYNMGWFR
QAPGQGLEAVASITWSGRDTFYADSVKGRFTISRDNSKNT
LYLQMNSLRAEDTAVYYCAANPWPVAAPRSGTYWGQGTLV
TVSSPSGQAGAAASESLFVSNHAYNWVNVISDLKKIEDLI
QSMHIDATLYTESDVHPSCKVTAMKCFLLELQVISLESGD
ASIHDTVENLIILANNSLSSNGNVTESGCKECEELEEKNI
KEFLQSFVHIVQMFINTSEASGGPEQMQLQESGPGLVKPS
ETLSLTCSVSGASISDSYWSWIRRSPGKGLEWIGYVHKSG
DTNYSPSLKSRVNLSLDTSKNQVSLSLVAATAADSGKYYC
ARTLHGRRIYGIVAFNEWFTYFYMDVWGNGTQVTVSSGGG
GSGGGGSGGGGSSDISVAPGETARISCGEKSLGSRAVQWY
QHRAGQAPSLIIYNNQDRPSGIPERFSGSPDSPFGTTATL
TITSVEAGDEADYYCHIWDSRVPTKWVFGGGTTLTVL
PGT121
SEQ ID NO: 8
QMQLQESGPGLVKPSETLSLTCSVSGASISDSYWSWIRRS
PGKGLEWIGYVHKSGDTNYSPSLKSRVNLSLDTSKNQVSL
SLVAATAADSGKYYCARTLHGRRIYGIVAFNEWFTYFYMD
VWGNGTQVTVSSGGGGSGGGGSGGGGSSDISVAPGETARI
SCGEKSLGSRAVQWYQHRAGQAPSLIIYNNQDRPSGIPER
FSGSPDSPFGTTATLTITSVEAGDEADYYCHIWDSRVPTK
WVFGGGTTLTVL
SEQ ID NO: 9
MGWSCIILFLVATATGVHSSLTQSPGTLSLSPGETAIISC
RTSQYGSLAWYQQRPGQAPRLVIYSGSTRAAGIPDRFSGS
RWGPDYNLTISNLESGDFGVYYCQQYEFFGQGTKVQVDIK
RTVAAPSVFIFPPSDEQLKSGTASVVCLLNNFYPREAKVQ
WKVDNALQSGNSQESVTEQDSKDSTYSLSSTLTLSKADYE
KHKVYACEVTHQGLSSPVTKSFNRGECGGGGSGGGGSEVQ
LVESGGELVQAGGSLRLSCAASGLTFSSYNMGWFRRAPGK
EREFVASITWSGRDTFYADSVKGRFTISRDNAKNTVYLQM
SSLKPEDTAVYYCAANPWPVAAPRSGTYWGQGTQVTVSSV
DEHHHHHHHHHH *
SEQ ID NO: 10
MGWSCIILFLVATATGVHSS
SEQ ID NO: 11
EVQLVESGGELVQAGGSLRLSCAASGLTFSSYNMGWFRRA
PGKEREFVASITWSGRDTFYADSVKGRFTISRDNAKNTVY
LQMSSLKPEDTAVYYCAANPWPVAAPRSGTYWGQGTQVTV
SSVDE
SEQ ID NO: 12
HHHHHHHHHH
SEQ ID NO: 13
MGWSCIILFLVATATGVHSQVRLSQSGGQMKKPGDSMRIS
CRASGYEFINCPINWIRLAPGKRPEWMGWMKPRHGAVSYA
RQLQGRVTMTRDMYSETAFLELRSLTSDDTAVYFCTRGKY
CTARDYYNWDFEHWGQGTPVTVSSASTKGPSVFPLAPSSK
STSGGTAALGCLVKDYFPEPVTVSWNSGALTSGVHTFPAV
LQSSGLYSLSSVVTVPSSSLGTQTYICNVNHKPSNTKVDK
KVEPKSCDK
SEQ ID NO: 14
MGWSCIILFLVATATGVHS
SEQ ID NO: 15
QVRLSQSGGQMKKPGDSMRISCRASGYEFINCPINWIRLA
PGKRPEWMGWMKPRHGAVSYARQLQGRVTMTRDMYSETAF
LELRSLTSDDTAVYFCTRGKYCTARDYYNWDFEHWGQGTP
VTVS
SEQ ID NO: 16
SASTKGPSVFPLAPSSKSTSGGTAALGCLVKDYFPEPVTV
SWNSGALTSGVHTFPAVLQSSGLYSLSSVVTVPSSSLGTQ
TYICNVNHKPSNTKVDKKVEPKSCDK
SEQ ID NO: 17
MGWSCIILFLVATATGVHSSLTQSPGTLSLSPGETAIISC
RTSQYGSLAWYQQRPGQAPRLVIYSGSTRAAGIPDRFSGS
RWGPDYNLTISNLESGDFGVYYCQQYEFFGQGTKVQVDIK
RTVAAPSVFIFPPSDEQLKSGTASVVCLLNNFYPREAKVQ
WKVDNALQSGNSQESVTEQDSKDSTYSLSSTLTLSKADYE
KHKVYACEVTHQGLSSPVTKSFNRGECGGGGSGGGGSQVQ
LVESGGGLVQPGGSLRLSCAASGLTFSSYNMGWFRQAPGQ
GLEAVASITWSGRDTFYADSVKGRFTISRDNSKNTLYLQM
NSLRAEDTAVYYCAANPWPVAAPRSGTYWGQGTLVTVSSV
DEHHHHHHHHHH
SEQ ID NO: 18
QVQLVESGGGLVQPGGSLRLSCAASGLTFSSYNMGWFRQA
PGQGLEAVASITWSGRDTFYADSVKGRFTISRDNSKNTLY
LQMNSLRAEDTAVYYCAANPWPVAAPRSGTYWGQGTLVTV
SS
SEQ ID NO: 19
MGWSCIILFLVATATGVHSQVRLSQSGGQMKKPGDSMRIS
CRASGYEFINCPINWIRLAPGKRPEWMGWMKPRHGAVSYA
RQLQGRVTMTRDMYSETAFLELRSLTSDDTAVYFCTRGKY
CTARDYYNWDFEHWGQGTPVTVSSASTKGPSVFPLAPSSK
STSGGTAALGCLVKDYFPEPVTVSWNSGALTSGVHTFPAV
LQSSGLYSLSSVVTVPSSSLGTQTYICNVNHKPSNTKVDK
KVEPKSCDKEGRGSLLTCGDVEENPGPMGWSCIILFLVAT
ATGVHSSLTQSPGTLSLSPGETAIISCRTSQYGSLAWYQQ
RPGQAPRLVIYSGSTRAAGIPDRFSGSRWGPDYNLTISNL
ESGDFGVYYCQQYEFFGQGTKVQVDIKRTVAAPSVFIFPP
SDEQLKSGTASVVCLLNNFYPREAKVQWKVDNALQSGNSQ
ESVTEQDSKDSTYSLSSTLTLSKADYEKHKVYACEVTHQG
LSSPVTKSFNRGECGGGGSGGGGSQVQLVESGGGLVQPGG
SLRLSCAASGLTFSSYNMGWFRQAPGQGLEAVASITWSGR
DTFYADSVKGRFTISRDNSKNTLYLQMNSLRAEDTAVYYC
AANPWPVAAPRSGTYWGQGTLVTVSSVDEHHHHHHHHHH
SEQ ID NO: 20
MKHLWFFLLLVAAPRWVLSQMQLQESGPGLVKPSETLSLT
CSVSGASISDSYWSWIRRSPGKGLEWIGYVHKSGDTNYSP
SLKSRVNLSLDTSKNQVSLSLVAATAADSGKYYCARTLHG
RRIYGIVAFNEWFTYFYMDVWGNGTQVTVSSASTKGPSVF
PLAPSSKSTSGGTAALGCLVKDYFPEPVTVSWNSGALTSG
VHTFPAVLQSSGLYSLSSVVTVPSSSLGTQTYICNVNHKP
SNTKVDKRVEEGRGSLLTCGDVEENPGPMAWTFLLLGLLS
HCTASVTSDISVAPGETARISCGEKSLGSRAVQWYQHRAG
QAPSLIIYNNQDRPSGIPERFSGSPDSPFGTTATLTITSV
EAGDEADYYCHIWDSRVPTKWVFGGGTTLTVLGQPKAAPS
VTLFPPSSEELQANKATLVCLISDFYPGAVTVAWKADSSP
VKAGVETTTPSKQSNNKYAASSYLSLTPEQWKSHKSYSCQ
VTHEGSTVEKTVAPTECSGGGGSGGGGSQVQLVESGGGLV
QPGGSLRLSCAASGLTFSSYNMGWFRQAPGQGLEAVASIT
WSGRDTFYADSVKGRFTISRDNSKNTLYLQMNSLRAEDTA
VYYCAANPWPVAAPRSGTYWGQGTLVTVSSVDEHHHHHHH
HHH
(HIV antibody light chain)
SEQ ID NO: 21
LTQSPGTLSLSPGETAIISCRTSQYGSLAWYQQRPGQAPR
LVIYSGSTRAAGIPDRFSGSRWGPDYNLTISNLESGDFGV
YYCQQYEFFGQGTKVQVDIKRTVAAPSVFIFPPSDEQLKS
GTASWCLLNNFYPREAKVQWKVDNALQSGNSQESVTEQDS
KDSTYSLSSTLTLSKADYEKHKVYACEVTHQGLSSPVTKS
FNRGEC
(HIV antibody Heavy chain)
SEQ ID NO: 22
QVRLSQSGGQMKKPGDSMRISCRASGYEFINCPINWIRLA
PGKRPEWMGWMKPRHGAVSYARQLQGRVTMTRDMYSETAF
LELRSLTSDDTAVYFCTRGKYCTARDYYNWDFEHWGQGTP
VTVSSASTKGPSVFPLAPSSKSTSGGTAALGCLVKDYFPE
PVTVSWNSGALTSGVHTFPAVLQSSGLYSLSSVVTVPSSS
LGTQTYICNVNHKPSNTKVDKKVEPKSCDK
SEQ ID NO: 23
GGGGSGGGGS
SEQ ID NO: 24
MKWVTFISLLFLFSSAYSQVQLVESGGGLVQPGGSLRLSC
AASGLTFSSYNMGWFRQAPGQGLEAVASITWSGRDTFYAD
SVKGRFTISRDNSKNTLYLQMNSLRAEDTAVYYCAANPWP
VAAPRSGTYWGQGTLVTVSSSGGGGSGGGGSGGGGSGGGG
SGNWVNVISDLKKIEDLIQSMHIDATLYTESDVHPSCKVT
AMKCFLLELQVISLESGDASIHDTVENLIILANNSLSSNG
NVTESGCKECEELEEKNIKEFLQSFVHIVQMFINTSGSTS
GSGKPGSGEGSTKGQVQLVQSGGQMKKPGESMRISCRASG
YEFIDCTLNWIRLAPGKRPEWMGWLKPRGGAVNYARPLQG
RVTMTRDVYSDTAFLELRSLTVDDTAVYFCTRGKNCDYNW
DFEHWGRGTPVIVSSGGGGSGGGGSGGGGSEIVLTQSPGT
LSLSPGETAIISCRTSQYGSLAWYQQRPGQAPRLVIYSGS
TRAAGIPDRFSGSRWGPDYNLTISNLESGDFGVYYCQQYE
FFGQGTKVQVDIKRVDEHHHHHHHHHH
SEQ ID NO: 25
QVQLVQSGGQMKKPGESMRISGRASGYEFIDCTLNWIRLA
PGKRPEWMGWLKPRGGAVNYARPLQGRVTMTRDVYSDTAF
LELRSLTVDDTAVYFCTRGKNCDYNWDFEHWGRGTPVIV
SEQ ID NO: 26
EIVLTQSPGTLSLSPGETAIISCRTSQYGSLAWYQQRPGQ
APRLVIYSGSTRAAGIPDRFSGSRWGPDYNLTISNLESGD
FGVYYCQQYEFFGQGTKVQVDIKR
SEQ ID NO: 27
LTQSPGTLSLSPGETAIISCRTSQYGSLAWYQQRPGQAPR
LVIYSGSTRAAGIPDRFSGSRWGPDYNLTISNLESGDFGV
YYCQQYEFFGQGTKVQVDIKRTVA
SEQ ID NO: 28
APSVFIFPPSDEQLKSGTASVVCLLNNFYPREAKVQWKVD
NALQSGNSQESVTEQDSKDSTYSLSSTLTLSKADYEKHKV
YACEVTHQGLSSPVTKSFNRGEC
SEQ ID NO: 29
MKWVTFISLLFLFSSAYSQVQLVESGGGLVQPGGSLRLSC
AASGLTFSSYNMGWFRQAPGQGLEAVASITWSGRDTFYAD
SVKGRFTISRDNSKNTLYLQMNSLRAEDTAVYYCAANPWP
VAAPRSGTYWGQGTLVTVSSSGGGGSGGGGSGGGGSGGGG
SGNWVNVISDLKKIEDLIQSMHIDATLYTESDVHPSCKVT
AMKCFLLELQVISLESGDASIHDTVENLIILANNSLSSNG
NVTESGCKECEELEEKNIKEFLQSFVHIVQMFINTSGSTS
GSGKPGSGEGSTKGSGEGSTKGEVQLVESGGGLVKPGGSL
RLSCSASGFDFDNAWMTWVRQPPGKGLEWVGRITGPGEGW
SVDYAAPVEGRFTISRLNSINFLYLEMNNLRMEDSGLYFC
ARTGKYYDFWSGYPPGEEYFQDWGRGTLVTVSSGGGGSGG
GGSGGGGSSYELTQETGVSVALGRTVTITCRGDSLRSHYA
SWYQKKPGQAPILLFYGKNNRPSGVPDRFSGSASGNRASL
TISGAQAEDDAEYYCSSRDKSGSRLSVFGGGTKLTVLLVD
EHHHHHHHHHH
SEQ ID NO: 30
SGEGSTKGEVQLVESGGGLVKPGGSLRLSCSASGFDFDNA
WMTWVRQPPGKGLEWVGRITGPGEGWSVDYAAPVEGRFTI
SRLNSINFLYLEMNNLRMEDSGLYFCARTGKYYDFWSGYP
PGEEYFQDWGRGTLVTV
SEQ ID NO: 31
SYELTQETGVSVALGRTVTITCRGDSLRSHYASWYQKKPG
QAPILLFYGKNNRPSGVPDRFSGSASGNRASLTISGAQAE
DDAEYYCSSRDKSGSRLSVFGGGTKLTVLL
SEQ ID NO: 32
MEQVQLVESGGGLVQPGGSLRLSCAASGLTFSSYNMGWFR
QAPGQGLEAVASITWSGRDTFYADSVKGRFTISRDNSKNT
LYLQMNSLRAEDTAVYYCAANPWPVAAPRSGTYWGQGTLV
TVSSPSGQAGAAASESLFVSNHAYNWVNVISDLKKIEDLI
QSMHIDATLYTESDVHPSCKVTAMKCFLLELQVISLESGD
ASIHDTVENLIILANNSLSSNGNVTESGCKECEELEEKNI
KEFLQSFVHIVQMFINTSEASGGPEQVQLVQSGAEVKKPG
ASVKVSCKASGYSFTGYTMNWVRQAPGQGLEWMGLITPYN
GASSYNQKFRGKATMTVDTSTSTVYMELSSLRSEDTAVYY
CARGGYDGRGFDYWGQGTLVTVSSGGGGSGGGGSSGGGSD
IQMTQSPSSLSASVGDRVTITCSASSSVSYMHWYQQKSGK
APKLLIYDTSKLASGVPSRFSGSGSGTDFTLTISSLQPED
FATYYCQQWSKHPLTFGQGTKLEIK
SEQ ID NO: 33
QVQLVQSGAEVKKPGASVKVSCKASGYSFTGYTMNWVRQA
PGQGLEWMGLITPYNGASSYNQKFRGKATMTVDTSTSTVY
MELSSLRSEDTAVYYCARGGYDGRGFDYWGQGTLVTVSSG
GGGSGGGGSSGGGSDIQMTQSPSSLSASVGDRVTITCSAS
SSVSYMHWYQQKSGKAPKLLIYDTSKLASGVPSRFSGSGS
GTDFTLTISSLQPEDFATYYCQQWSKHPLTFGQGTKLEIK
SEQ ID NO: 34
MEQVQLVESGGGLVQPGGSLRLSCAASGLTFSSYNMGWFR
QAPGQGLEAVASITWSGRDTFYADSVKGRFTISRDNSKNT
LYLQMNSLRAEDTAVYYCAANPWPVAAPRSGTYWGQGTLV
TVSSPSGQAGAAASESLFVSNHAYNWVNVISDLKKIEDLI
QSMHIDATLYTESDVHPSCKVTAMKCFLLELQVISLESGD
ASIHDTVENLIILANNSLSSNGNVTESGCKECEELEEKNI
KEFLQSFVHIVQMFINTSEASGGPEQVQLVQSGAEVKKPG
ASVKVSCKASGYSFTGYTMNWVRQAPGQGLEWMGLITPYN
GASSYNQKFRGKATMTVDTSTSTVYMELSSLRSEDTAVYY
CARGGYDGRGFDYWGQGTLVTVSSGGGGSGGGGSSGGGSD
IQMTQSPSSLSASVGDRVTITCSASSSVSYMHWYQQKSGK
APKLLIYDTSKLASGVPSRFSGSGSGTDFTLTISSLQPED
FATYYCQQWSKHPLTFGQGTKLEIKEPKSSDKTHTSPPSP
ELDIVLSQSPAIMSASPGEKVTISCSASSSVSYMYWYQQK
PGSSPKPWIYRTSNLASGVPARFSGSGSGTSYSLTISSME
AEDAATYYCQQYHSYPPTFGAGTKLELKSSGGGGSGGGGG
GSSRSSLEVKLVESGPELKKPGETVKISCKASGYTFTDYS
MHWVNQAPGKGLKWMGWINTETGEPSYADDFKGRFAFSLE
TSASTAYLQINNLKNEDTATYFCATDYGDYFDYWGQGTTL
TVSSAKTTPPSVTS
SEQ ID NO: 35
QVQLVQSGAEVKKPGASVKVSCKASGYSFTGYTMNWVRQA
PGQGLEWMGLITPYNGASSYNQKFRGKATMTVDTSTSTVY
MELSSLRSEDTAVYYCARGGYDGRGFDYWGQGTLVTVSSG
GGGSGGGGSSGGGSDIQMTQSPSSLSASVGDRVTITCSAS
SSVSYMHWYQQKSGKAPKLLIYDTSKLASGVPSRFSGSGS
GTDFTLTISSLQPEDFATYYCQQWSKHPLTFGQGTKLEIK
EPKSSDKTHTSPPSPELDIVLSQSPAIMSASPGEKVTISC
SASSSVSYMYWYQQKPGSSPKPWIYRTSNLASGVPARFSG
SGSGTSYSLTISSMEAEDAATYYCQQYHSYPPTFGAGTKL
ELKSSGGGGSGGGGGGSSRSSLEVKLVESGPELKKPGETV
KISCKASGYTFTDYSMHWVNQAPGKGLKWMGWINTETGEP
SYADDFKGRFAFSLETSASTAYLQINNLKNEDTATYFCAT
DYGDYFDYWGQGTTLTVSSAKTTPPSVTS
SEQ ID NO: 36
MEVQLVESGGGVVRPGGSLRLSCAASGFTFDDYGMSWVRQ
APGKGLEWVSGINWNGGSTGYADSVKGRFTISRDNAKNSL
YLQMNSLRAEDTAVYYCARGRSLLFDYWGQGTLVTVSRGG
GGSGGGGSGGGGSSELTQDPAVSVALGQTVRITCQGDSLR
SYYASWYQQKPGQAPVLVIYGKNNRPSGIPDRFSGSSSGN
TASLTITGAQAEDEADYYCNSRDSSGNHVVFGGGTKLTVL
PSGQAGAAASESLFVSNHAYNWVNVISDLKKIEDLIQSMH
IDATLYTESDVHPSCKVTAMKCFLLELQVISLESGDASIH
DTVENLIILANDSLSSNGNVTESGCKECEELEEKNIKEFL
QSFVHIVQMFINTSEASGGPEQVQLVQSGAEVKKPGASVK
VSCKASGYSFTGYTMNWVRQAPGQGLEWMGLITPYNGASS
YNQKFRGKATMTVDTSTSTVYMELSSLRSEDTAVYYCARG
GYDGRGFDYWGQGTLVTVSSGGGGSGGGGSSGGGSDIQMT
QSPSSLSASVGDRVTITCSASSSVSYMHWYQQKSGKAPKL
LIYDTSKLASGVPSRFSGSGSGTDFTLTISSLQPEDFATY
YCQQWSKHPLTFGQGTKLEIK
SEQ ID NO: 37
MKWVTFISLLFLFSSAYSQVQLVESGGGLVQPGGSLRLSC
AASGLTFSSYNMGWFRQAPGQGLEAVASITWSGRDTFYAD
SVKGRFTISRDNSKNTLYLQMNSLRAEDTAVYYCAANPWP
VAAPRSGTYWGQGTLVTVSSSGGGGSGGGGSGGGGSGGGG
SGNWVNVISDLKKIEDLIQSMHIDATLYTESDVHPSCKVT
AMKCFLLELQVISLESGDASIHDTVENLIILANNSLSSNG
NVTESGCKECEELEEKNIKEFLQSFVHIVQMFINTSGSTS
GSGKPGSGEGSTKGQHLVQSGTQVKKPGASVRISCQASGY
SFTDYVLHWWRQAPGQGLEWMGWIKPVYGARNYARRFQGR
INFDRDIYREIAFMDLSGLRSDDTALYFCARDGSGDDTSW
HLDPWGQGTLVIVSAASTKGGGGGSGGGGSGGGGSDIQMT
QSPSSLSASVGDRVTITCQAGQGIGSSLQWYQQKPGKAPK
LLVHGASNLHRGVPSRFSGSGFHTTFSLTISGLQRDDFAT
YFCAVLEFFGPGTKVEIKRTVAAPSVDEHHHHHHHHHH
SEQ ID NO:
QVQLVESGGGLVQPGGSLRLSCAASGLTFSSYNMGWFRQA
PGQGLEAVASITWSGRDTFYADSVKGRFTISRDNSKNTLY
LQMNSLRAEDTAVYYCAANPWPVAAPRSGTYWGQGTLVTV
SS
SEQ ID NO: 3
QHLVQSGTQVKKPGASVRISCQASGYSFTDYVLHWWRQAP
GQGLEWMGWIKPVYGARNYARRFQGRINFDRDIYREIAFM
DLSGLRSDDTALYFCARDGSGDDTSWHLDPWGQGTLVIVS
AASTK
SEQ ID NO:
DIQMTQSPSSLSASVGDRVTITCQAGQGIGSSLQWYQQKP
GKAPKLLVHGASNLHRGVPSRFSGSGFHTTFSLTISGLQR
DDFATYFCAVLEFFGPGTKVEIKRTVAAPS

1. A compound comprising:
an NK engaging domain comprising SEQ ID NO:11; SEQ ID NO:18; or amino acids 1-240 of SEQ ID NO:5;
an NK activating domain operably linked to the NK engaging domain comprising IL-15 or a functional fragment thereof; and
a targeting domain that selectively binds to a viral antigen and is operably linked to the NK activating domain and the NK engaging domain, wherein the viral antigen is an HIV antigen.

2. (canceled)

3. The compound of claim 1, wherein the viral antigen is present on an infected cell.

4-5. (canceled)

6. The compound of claim 1, wherein the NK engaging domain moiety comprises an antibody or a binding fragment thereof or a nanobody.

7. The compound of claim 6, wherein the antibody fragment comprises an scFv, a F(ab)2, or a Fab.

8. The compound of claim 6, wherein the antibody or a binding fragment thereof or the nanobody is human, humanized, or camelid.

9. The compound of claim 6, wherein the antibody or a binding fragment thereof or the nanobody is camelid.

10. The compound of claim 6, wherein the IL-15 comprises an amino acid sequence of SEQ ID NO: 4 or a functional variant thereof

11. The compound of claim 10, wherein the functional variant of IL-15 comprises an N72D or N72A amino acid substitution as compared to SEQ ID NO:4.

12. The compound of claim 1, wherein the targeting domain moiety comprises an antibody or a binding fragment thereof or a nanobody.

13. The compound of claim 12, wherein the antibody binding fragment comprises an scFv, a F(ab)2, or a Fab.

14-16. (canceled)

17. The compound of claim 1, comprising at least one flanking sequence linking two of the domains.

18. The compound of claim 17, further comprising a second flanking sequence linking the two linked domains with the third domain.

19. The compound of claim 18, wherein the flanking sequences flank the NK activating domain.

20. The compound of claim 18, wherein a first flanking sequence is C-terminal to the NK engaging domain and wherein a second flanking sequence is N-terminal to the anti-viral targeting domain.

21. The compound of claim 1, further comprising a second targeting domain.

22. The compound of claim 1, further comprising a second NK engaging domain.

23. The compound of claim 1, further comprising a second NK activating domain.

24. The compound of claim 1, wherein the compound is SEQ ID NO:5, 7, 24, 29 or 37.

25. A composition comprising:
the compound of claim 1; and
a pharmaceutically acceptable carrier.

26. A method comprising:
administering to a subject the compound of claim 1 in an amount effective to induce NK-mediated killing of a target cell.

27. The method of claim 26, wherein the target cell is infected with HIV.

28-29. (canceled)

30. A method for stimulating expansion of NK cells in vivo, the method comprising:
administering to a subject an amount of the compound of claim 1 effective to stimulate expansion of NK cells in the subject.

31. The method of claim 30, wherein the subject is infected with HIV.

32-33. (canceled)

34. A method of treating viral infection in a subject, the method comprising:
administering to the subject an amount of the compound of claim 1 effective for treating the viral infection.

35. The method of claim 34, wherein the subject is infected with HIV.

36. (canceled)

37. An isolated nucleic acid sequence of SEQ ID NO:6.

38. An isolated amino acid sequence of SEQ ID NO:7.

39. An isolated amino acid sequence comprising the sequence of camCD16/IL-15/SEQ ID NO:8.

40. An isolated amino acid sequence comprising SEQ ID NO:5, 9, 17, 27, 28, 13, 15, 16, 17, 18, 19, 20.

41. The isolated amino acid of claim 40, further comprising an isolated amino acid sequence of SEQ ID NO:10.

42. An isolated amino acid sequence comprising SEQ ID NO:18 operably linked to IL-15.

43. An isolated amino acid sequence of SEQ ID NO:20-26.

44. A method of making the compound of claim 1 comprising:
co-transfecting into mammalian cells a first polynucleotide comprising a nucleotide sequence encoding an amino acid sequence comprising an immunoglobulin heavy chain of SEQ ID NO:22, 25, 30 or 39 and a second polynucleotide comprising a nucleotide sequence encoding an amino acid sequence comprising an immunoglobulin light chain of SEQ ID NO:21, 26, 31 or 40, respectively; and
(ii) collecting a supernatant from the mammalian cells, wherein the resulting compound binds to a viral antigen.

45. The method of claim 44, wherein the viral antigen is derived from HIV.

46. The method of claim 45, wherein the viral antigen is Env.

47. An isolated DNA sequence encoding the amino acid sequences of SEQ ID NOs:21, 22, 25, 26, 30, 31, 39 or 40.

48. A pharmaceutical composition comprising SEQ ID NO:7, 24, 29, 32, 34, 36 and 37 in a pharmaceutically acceptable carrier.

49. A method of treating a subject comprising administering to the subject a pharmaceutical composition comprising SEQ ID NO:5, 7, 24, 29, 32, 34, 36 and 37 in a pharmaceutically acceptable carrier.

50. A method of treating a subject having or being at risk for developing AIDS, comprising administering to the subject a pharmaceutical composition comprising SEQ ID NO:5, 7, 24, 29, and 37.
